(19) United States
(12) Patent Application Publication
(10) Pub. No.: US 2003/0104475 A1
(43) Pub. Date: Jun. 5, 2003

(54) Method and apparatus for computer modeling of an adaptive immune response

(76) Inventors: Scott D. Kelly, Champaign, IL (US); David J. Klinke II, San Bruno, CA (US); Clement Leong, Erskineville (AU); Annette K. Lewis, Menlo Park, CA (US); Miles S. Okino, Belmont, CA (US); Thomas S. Paterson, West Hollywood, CA (US); Lisl K.M. Shoda, Redwood City, CA (US); Cynthia Stokes, Menlo Park, CA (US); Herbert K. Struemper, Menlo Park, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/186,938

(22) Filed: Jun. 28, 2002

Related U.S. Application Data
(60) Provisional application No. 60/301,278, filed on Jun. 28, 2001.

(51) Int. Cl. G01N 33/53; G01N 33/50

(52) U.S. Cl. 435/7.1; 435/4; 702/19

(57) ABSTRACT

The present invention relates generally to a computer model of an adaptive immune response. One embodiment of the invention relates to a computer model of an adaptive immune response within the framework of signals conveyed at the site of antigen exposure. Another embodiment of the model includes a representation of complex physiological regulatory mechanisms related to, for example, cellular dynamics, mediator production, antigen-presenting cell (APC) recruitment, APC maturation, lymphocyte activation, lymphocyte trafficking, and/or lymphocyte effector function. In another embodiment, the model can account for mediator production in response to antigen within a chronically inflamed peripheral tissue, as well as the regulatory effects of mediators on APC and lymphocyte population dynamics, including maturation, activation, and apoptosis, and the regulatory effects of mediators produced by APCs and lymphocytes on a chronically inflamed peripheral tissue. Another embodiment of the invention relates to an analytical model of an adaptive immune response.

### CROSS-REFERENCE TO RELATED APPLICATION

The present invention is related to and claims priority under 35 U.S.C. §119(e) to U.S. Provisional Patent Application Serial No. 60/301,278, filed Jun. 28, 2001, entitled “Method and Apparatus for Computer Modeling of T Cells,” the specification of which is incorporated herein by reference.

### COPYRIGHT NOTICE

A portion of the disclosure of the patent document contains material that is subject to copyright protection. The copyright owner has no objection to the facsimile reproduction by anyone of the patent document of the patent disclosure, as it appears in the Patent and Trademark Office patent file or records, but otherwise reserves all copyright rights whatsoever.

### BACKGROUND OF THE INVENTION

The present invention relates generally to a computer model of the adaptive immune response. In one embodiment, the present invention relates to a computer model of the adaptive immune response within the framework of signals conveyed at the site of antigen exposure, where the signals include signals impacting antigen-presenting cells (APCs) and signals delivered by APCs and by responding lymphocytes. In another embodiment, the present invention relates to an analytical model of an adaptive immune response.

The human immune system has evolved as a complex process by which it is able to identify and respond to a vast array of genetically, biochemically, and behaviorally distinct microbial pathogens while not responding to the vast array of innocuous environmental elements or to the vast array of normal human cellular and biochemical elements. Adaptive immune responses involve populations of specialized immune cells or lymphocytes that have evolved to match the wide array of elements they may encounter. Particularly, lymphocyte populations are composed of a large number of individual cells, where each lymphocyte expresses receptors with a distinct molecular sequence of a distinct affinity and specificity. As a result, each lymphocyte only binds the molecular sequence, or antigen(s), that molecularly interact with its receptor, and these cells are therefore classified as antigen-specific.

The nature of the adaptive immune response is to selectively activate and expand antigen-specific lymphocytes if and only if the antigens are presented in the correct context. The dual requirements that an antigen-specific cell must not only recognize its particular antigen(s), but must also recognize the antigen within a particular enabling context, prevents lymphocyte responses to self or innocuous environmental antigens and promotes lymphocyte responses to pathogens. The enabling context is largely determined at the site of antigen exposure (i.e., peripheral tissue), which is generally anatomically separate from the site of lymphocyte expansion (i.e., secondary lymphoid tissue).

APCs take up and process antigen at the site of antigen exposure and are primarily responsible for the transport of antigen from the site of antigen exposure to the site of lymphocyte expansion. Critical signals, directed to the APCs at the site of antigen exposure, shape the manner in which APCs subsequently present antigen to lymphocytes. The manner of APC antigen presentation, in combination with other signals present in the secondary lymphoid tissue, is responsible for inducing a lymphocyte response and in guiding the character of the response. For example, mediator production, costimulatory molecule expression, and antigen presentation by APCs varies according to the peripheral environment in which antigen is taken up and significantly contributes to the development of a T helper (Th) 1 or Th2 biased T lymphocyte response.

Once activated, lymphocytes may directly or indirectly drive a cellular or humoral adaptive immune response. In the case of an infecting pathogen, a cellular response is characterized by antigen-specific lymphocytes that traffic to the site of pathogen exposure. The lymphocytes, which recognize antigen derived from the pathogen, may then directly kill the pathogen or activate other immune cells to kill the pathogen. A humoral response is characterized by antigen-specific lymphocytes that generate an antigen-specific antibody response; the antibodies bind the pathogens and facilitate their clearance from the body. As described above for Th1 and Th2 lymphocyte responses, the generation of cellular and/or humoral adaptive immune responses is largely guided by a combination of antigen and context.

The adaptive immune response also generates memory lymphocytes, which allow the immune system to increase its response efficiency. In the case of an infecting pathogen, these antigen-specific long-lived lymphocytes remain in the body after the pathogen is cleared, such that in later encounters with the same pathogen, the immune system responds more quickly and with greater strength than in the first encounter. Immune memory is generated throughout the lifespan of an individual and confers the advantage that secondary exposure to a particular bacterium, virus, parasite, or fungus, can be cleared by the immune system with minimal compromise in the individual's ability to function.

Because the immune system must be able to respond to the vast array of pathogens that may be encountered but should not respond to the even wider array of innocuous environmental elements or self-elements, the adaptive immune response is a tightly regulated process. However, there is clearly a potential for inappropriate immune responses, as represented by the existence of allergic and autoimmune diseases.

The etiologies of inappropriate immune responses that manifest as allergic diseases (e.g., asthma, allergic rhinitis, food allergy) are unproven but likely include genetic factors, history of exposure to environmental elements, and history of exposure to pathogens. The result is an inappropriate adaptive immune response to a normally innocuous environmental element (antigen), leading to elevated levels of immunoglobulin (Ig) E and chronic inflammation at the exposure site. The development of pharmaceutical treatments for these diseases has historically focused on controlling the symptoms of disease. However, as our understanding of immune processes improves, some newer treatments have been directed towards modifying the underlying inappropriate immune response. This effort has been complicated by the fact that the adaptive response is highly complex, highly redundant, and tightly regulated, making the selection of appropriate intervention sites difficult.

The etiologies of inappropriate immune responses that manifest as autoimmune diseases (e.g., rheumatoid arthritis, multiple sclerosis, inflammatory bowel disease) are unproven but likely include genetic factors, history of exposure to environmental elements, and history of exposure to pathogens. The result is an inappropriate adaptive immune response to a self-molecule (antigen), leading to chronic inflammation and targeted tissue destruction at the exposure site. As discussed above with allergic diseases, emerging pharmaceutical therapies are directed towards modifying underlying inappropriate immune responses. However, the development of these new therapies has been complicated by the intricacies of the immune system and the need to selectively target inappropriate responses, while leaving appropriate immune responses intact.

Several researchers have constructed simple mathematical models of antigen-specific lymphocyte expansion and its control by cytokines or antigen abundance (De Boer et al., J. Virol., 75:10663-10669, 2001; Louzon et al., J. Autoimmunity, 17:311-321, 2001; Yates et al., J. Theor. Biol., 206:539-200, 2000; Fishman & Perelson, Bull. Math. Biol., 61:403-436, 1999). These models were largely restricted to lymphocyte responses and did not represent important interactions that take place at the site of primary antigen exposure and that largely determine the enabling context for lymphocyte expansion. Specifically, these models did not include detailed representations of APC populations or the influence of particular peripheral tissues on the APCs. In addition, these models did not represent important feedback pathways from expanded lymphocyte populations to the site of antigen exposure; wherein, antigen-specific lymphocytes traffic from a lymphoid tissue to the site of antigen exposure to directly or indirectly act against the antigen source.

Because these existing models do not include all aspects of the adaptive immune response, there is a need to develop a more comprehensive model of the adaptive immune response. One embodiment of the invention disclosed herein is a computer model of the adaptive immune response within the framework of signals conveyed at the site of antigen exposure, where the signals include signals received by antigen-presenting cells (APCs) and signals delivered by APCs and by responding lymphocytes.

### SUMMARY OF THE INVENTION

The present invention relates generally to a computer model of an adaptive immune response. One embodiment of the invention relates to a computer model of an adaptive immune response within the framework of signals conveyed at the site of antigen exposure. Another embodiment includes a representation of complex physiological regulatory mechanisms related to, for example, antigen-presenting cell (APC) recruitment, APC maturation, lymphocyte activation, and/or lymphocyte trafficking.

In one embodiment, the model can account for cellular dynamics and mediator production in response to antigen within a chronically inflamed peripheral tissue, as well as the regulatory effects on APC, APC population dynamics and activities, and lymphocyte population dynamics, including maturation, activation, effector function, and apoptosis. In addition, the model can account for immune cell trafficking between a chronically inflamed peripheral tissue and secondary lymphoid tissues. In this embodiment, the model can simulate a diverse set of adaptive immune responses, from acute to chronic progressive, and can predict the likely effects of therapeutic inventions.

In another embodiment, a tolerant immune reaction can be modeled; wherein, signals and characteristics of the peripheral tissue do not provoke a lymphocyte response to a particular antigen.

Another embodiment of the invention is an analytical model of the adaptive immune response.

### BRIEF DESCRIPTION OF THE DRAWINGS

FIG. 1 is a schematic representation of a computer system within which software for performing the methods of the invention may reside or be executed.

FIG. 2 is a summary diagram that depicts the components of a computer model and their interconnectedness, according to an embodiment of the present invention where the airway is an example of a peripheral tissue.

FIG. 3 depicts a flowchart for a method for developing a computer model of an adaptive immune response according to one embodiment of the invention.

FIG. 4 depicts a flowchart for a method for developing a computer model of an adaptive immune response according to another embodiment of the invention.

FIG. 5A shows a portion of the Effect Diagram representing dendritic cell (DC) precursors migrating from the blood into the airway and airway DC subpopulations undergoing maturation, according to an embodiment of the present invention.

FIG. 5B shows a portion of the Effect Diagram representing DC subpopulations trafficking between the airway and lymph nodes and DCs in the lymph node, according to an embodiment of the present invention.

FIG. 6A shows a portion of the Effect Diagram representing the biological processes involved in migration and maturation of airway DCs, according to an embodiment of the present invention.

FIG. 6B shows a portion of the Effect Diagram representing the biological processes involved in maturation and migration of lymph node DCs, according to an embodiment of the present invention.

FIG. 7A shows a portion of the Effect Diagram representing the biological processes involved in antigen presentation by airway DCs, according to an embodiment of the present invention.

FIG. 7B shows a portion of the Effect Diagram representing the biological processes involved in antigen presentation by lymph node DCs, according to an embodiment of the present invention.

FIG. 8 shows a portion of the Effect Diagram representing the biological processes of mediator production by airway and lymph node DCs, according to an embodiment of the present invention.

FIG. 9A shows a portion of the Effect Diagram representing the biological processes that determine IL-12 homodimer and heterodimer production capability of maturing airway DCs, according to an embodiment of the present invention.

FIG. 9B shows a portion of the Effect Diagram representing the biological processes that determine IL-12 homodimer and heterodimer production capability of DCs trafficking between the airway and lymph nodes and maturing lymph node DCs, according to an embodiment of the present invention.

FIG. 9C, in conjunction with FIG. 9D, shows a portion of the Effect Diagram representing the biological processes involved in late-stage regulation of IL-12 homodimer and heterodimer production capability in mature DCs, according to an embodiment of the present invention.

FIG. 9D, in conjunction with FIG. 9C, shows a portion of the Effect Diagram representing the biological processes involved in late-stage regulation of IL-12 homodimer and heterodimer production capability in mature DCs, according to an embodiment of the present invention.

FIG. 10 shows an example of an Effect Diagram that displays a taxonomy of CD4+ T lymphocyte states, according to an embodiment of the present invention.

FIG. 11 shows an example of a portion of an Effect Diagram that calculates CD4+ T lymphocyte cytokine production, according to an embodiment of the present invention.

FIG. 12 shows an example of a portion of an Effect Diagram relating cytokine binding and cell-cell interactions to CD4+ T lymphocyte expansion, differentiation, and apoptosis, according to an embodiment of the present invention.

FIG. 13 shows the Effect Diagram of FIG. 12 with an example of the mathematical calculation contained in the LN pe2 expansion function node, according to an embodiment of the present invention.

FIG. 14 illustrates an Effect Diagram that depicts Th1 and Th2 cell trafficking as modulated by chemokines and adhesion molecules according to an embodiment of the present invention where the airway (AW) is an example of a peripheral tissue.

FIG. 15 illustrates an output of the model showing expansion of Th1 (♦) and Th2 cells (●) as a function of antigen dose after simulated in vitro primary culture, according to an embodiment of the present invention.

FIG. 16 illustrates an output of the model that depicts evolving numbers of Th1 (_) and Th2 (_) cells during a simulated in vitro primary culture at relatively low antigen dose, according to an embodiment of the present invention.

FIG. 17 illustrates an output of the model that depicts evolving numbers of Th1 (_) and Th2 (_) cells during a simulated in vitro primary culture at a lower antigen dose than used in FIG. 16, according to an embodiment of the present invention.

FIG. 18 illustrates an Effect Diagram that depicts the recruitment of DC precursor populations from the blood into the peripheral tissue according to an embodiment of the present invention where the airway is an example of peripheral tissue.

FIG. 19 illustrates an output of the model that depicts the simulated kinetics of adhesion molecule expression in the airway tissue following antigen challenge at time equal to zero, according to an embodiment of the present invention.

FIG. 20 illustrates an output of the model that depicts the simulated kinetics of blood monocytes (_)compared with the experimental results reported by Whitelaw 1966 (●), according to an embodiment of the present invention.

FIG. 21 illustrates an output of the model that depicts the simulated kinetics of tissue DCs (_) compared with the experimental results reported by Holt et al. 1994 (●), according to an embodiment of the present invention.

FIG. 22 illustrates an output of the model that depicts the kinetics of blood monocytes (_), blood DC (_ _), and lung DC (_ ▪_) responses following antigen challenge at time equal to zero compared with the experimental results reported by Upham et al. 1999 (blood monocytes=o; blood DCs=x) and McWilliam et al. 1994 (lung DCs=+), according to an embodiment of the present invention.

FIG. 23 illustrates an output of the model that depicts the simulated kinetics of DC migration to the lymph node in the context of non-productive interactions with CD4+ T lymphocytes (₁₃ ) compared with the experimental results reported by Ingulli et al. 1997 (●) and Vermaelen et al. 2001 (▪), according to an embodiment of the present invention.

FIG. 24 illustrates an output of the model that depicts the simulated kinetics of DC migration to the lymph node in the context of productive interactions with CD4+ T lymphocytes (_) compared with the experimental results reported by Ingulli et al. 1997 (●), according to an embodiment of the present invention.

### DETAILED DESCRIPTION

### Overview

The present invention relates to computer modeling of an adaptive immune response. The adaptive immune response model can be used in isolation or integrated with other components to represent a healthy or diseased physiological system whose state is affected by the adaptive immune response. Embodiments of the present invention relate to modeled responses of antigen-presenting cells (APCs) and lymphocytes to immunogenic stimuli in the context of human diseases that involve the adaptive immune response (e.g., allergic asthma). In particular, one embodiment of the model includes a peripheral tissue environment, a lymphoid tissue environment, and traffic of immune cells between the two compartments. The peripheral tissue represented in the model can include for example, lung, skin, intestine, joint, or the central nervous system. The term lymphoid tissue environment as used herein can include primary, secondary, and tertiary lymphoid tissues.

The model can further include the character and kinetics of antigen exposure, the character and dynamics of APC populations (e.g., dendritic cells), the expansion, differentiation and contraction of antigen-specific lymphocyte populations (e.g., T lymphocytes), and the creation and maintenance of memory lymphocyte populations.

In one embodiment, the model includes one or more of the following features: (1) communication between a peripheral tissue and a secondary lymphoid tissue, (2) establishing a stable balance of populations within and between the two compartments, and (3) feedback pathways that allow for progressive changes in a disease state (i.e., the model is not limited to a steady-state representation).

The computer model of the present invention can be used to identify pharmaceutical interventions to treat immune diseases such as allergic asthma. In another embodiment, therapies affecting pathways that are present in the model can be implemented and used to predict therapeutic outcomes.

### Mathematical Model

The mathematical model implemented by the computer-executable software code represents the dynamic biological processes related to an adaptive immune response. The form of the mathematical equations employed may include, for example partial differential equations, stochastic differential equations, differential algebraic equations, difference equations, cellular automata, coupled maps, equations of networks of Boolean or fuzzy logical networks, etc. In one embodiment, the forms of the mathematical equations used in the model are ordinary differential equations:

dx/dt=f(x, p, t),

where x is an N dimensional vector whose elements represent the biological variables of the system (for example concentrations of chemical mediators, monocyte density in the blood, naïve T cell density in lymphoid tissue, etc.), t is time, dx/dt is the rate of change of x, p is an M dimensional set of system parameters (for example sensitivity of the blood monocyte recruitment rate to P-selectin, naïve T cell efflux rate, equilibrium dissociation constant for IL-10, etc.), and f is a function that represents the complex interactions among biological variables.

The term “biological variables” refers to the biological constituents that make up a biological process. Mathematically, the above x represents the biological variables in the model. For example, the biological variables can include metabolites, DNA, RNA, proteins, enzymes, hormones, cells, organs, tissues, portions of cells, tissues, or organs, subcellular organelles, chemically reactive molecules like H⁺, superoxides, ATP, citric acid, protein albumin, as well as combinations or aggregate representations of these types of biological variables. In addition, biological variables can include response-provoking agents, such as antigen or methacholine, and therapeutic agents such as steroids, β-agonists, or leukotriene antagonists.

The term “parameter” is used herein to mean a number that characterizes the behavior of a single biological variable or the interaction between two or more biological variables. For example, a parameter could be the baseline synthesis of a mediator, baseline expression of a cell surface molecule, or the maximum number of lymphocytes that may interact with any one APC. Parameters may also be used to specify synthetic or environmental factors, as well as intrinsic biological properties.

The term “biological process” is defined herein to mean an interaction or series of interactions between biological variables. Biological processes can include, for example, cellular recruitment, regulation of maturation, induction of anergy, or regulation of chemical mediator production. Each biological variable of the biological process can be influenced, for example, by at least one other biological variable in the biological process by some biological mechanism, which need not be specified or even understood.

The term “biological state” is used herein to mean the result of the occurrence of a series of biological processes. As the biological processes change relative to each other, the biological state also undergoes changes. One measurement of a biological state, is the level of activity of biologic variables, parameters, and/or processes at a specified time and under specified experimental or environmental conditions.

In one embodiment the biological state can be mathematically defined by the values of x and p at a given time. Once a biological state of the model is mathematically specified, numerical integration of the above equation using a computer determines, for example, the time evolution of the biological variables x(t) and hence the evolution of the biological state over time.

A biological state can include, for example, the state of an individual cell, a population of cells, a tissue, and/or a multi-cellular organism. A biological state can also include the state of a mediator concentration in the plasma, interstitial fluid, or intracellular fluid. For example, a biological state of an APC population can include the APC density or the antigen-presenting capacity in a particular peripheral tissue of a particular patient type at a particular point in time.

Adaptive immune responses are the set of responses that target specific antigen for immune activity and that are mounted by the immune system. In one embodiment of the invention, the biological state modeled is the state of an adaptive immune response. The term “adaptive immune response” as used herein comprises a combination of at least two of the following classes: biological processes at the site of antigen exposure, the impact of these biological processes on the character or behavior of immune cells, biological processes relating to cellular dynamics between a site of antigen exposure and lymphoid tissue, biological processes relating to a primary lymphoid tissue, biological processes relating to the interaction between APCs and lymphocytes, or biological processes relating to the feedback of immune cells on biological processes at the site of antigen exposure.

For example, the site of antigen exposure can include a peripheral tissue where the biological processes of the tissue can include invasion of host cells by a pathogen, interactions among inflammatory cells, or mediator levels. The impact of biological processes on immune cells might include, for example, the impact of gamma interferon production and binding on the ability of APCs to produce interleukin 12. Cellular dynamics between a site of antigen exposure and a lymphoid tissue might include for example, trafficking of APCs through the lymphatics or trafficking of lymphocytes through the blood. Feedback of immune cells on biological processes at the site of antigen exposure might include, for example, T lymphocyte activation of peripheral tissue macrophages. In one embodiment, a model of the adaptive immune response includes representation of at least two biological compartments including a peripheral site of antigen exposure and a lymphoid tissue, representation of the biological processes at a peripheral site and their impact on APCs and lymphocytes, representation of the cellular dynamics between a peripheral site and a lymphoid tissue, and representation of the impact of immune cells on a peripheral site.

The model of the adaptive immune response could be integrated with any number of other components to represent a healthy or diseased biological state. The method of this integration would involve modifying the peripheral tissue to assume the attributes of the peripheral tissue targeted in a particular biological state. In one embodiment, characteristics of cell types, cellular abnormalities, physiological abnormalities, and the chemical mediator environment of the peripheral tissue can be modeled appropriately. In another embodiment, the adaptive immune response model can be modified to reflect the nature of APCs and lymphocytes that associated with a biological state. The regions of interface can, for example, include modulation of antigen-presenting cell function by the peripheral tissue and modulation of the peripheral tissue by the antigen-presenting cells and the antigen-specific lymphocytes.

The term “simulation” is used herein to mean solution of a mathematical model by the numerical or analytical methods. For example, simulation can mean the numerical integration of the mathematical model of the biological state defined by the above equation, (i.e. dx/dt=f(x, p, t)) and specifying an initial value of x.

The term “disease state” is used herein to mean a biological state where one or more biological processes are related to the cause(s) or the clinical signs of the disease. For example, a disease state can be the state of a diseased cell, a diseased organ, a diseased tissue, or a diseased multi-cellular organism. Such diseases can include, for example, acquired immune deficiency syndrome, delayed-type hypersensitivity, systemic anaphylaxis, allergic asthma, cancer, inflammatory bowel disease, systemic lupus erythematosus, multiple sclerosis, type I diabetes, and rheumatoid arthritis. A diseased multi-cellular organism can be, for example, an individual patient, a specific group of human patients, or the general human population as a whole. A diseased state could refer to, for example, a diseased protein such as a defective interferon-gamma receptor or a diseased process, such as defects in cellular activation, cell signaling, or cell mediator production, which may occur in several different organs.

The term “biological attribute” is used herein to mean observations or diagnostic criteria associated with a biological state. The biological attributes of a biological state can be measurements of biological variables, parameters, and/or processes. For example, for the disease allergic asthma, the biological attributes associated with the adaptive immune response can include APC dynamics, T lymphocyte dynamics, or T lymphocyte cytokine production.

The term “simulated biological attribute” is used herein to mean measurements on model variables or processes corresponding to biological attributes. For example, simulated biological attributes associated with the modeled adaptive immune response might include measurements of APC or T lymphocyte dynamics.

The term “substantially consistent” is used herein to mean that the relationship between simulated biological attributes and biological attributes is sufficiently similar to conclude that a simulated biological attribute accurately represents a biological attribute; the simulated biological attribute and biological attribute do not have to be identical. The term “substantially consistent” can be, for example, simulation outcomes demonstrating relative changes in a pattern of cytokine levels that are similar to relative changes in a pattern of cytokine levels measured in an in vitro experiment but with different absolute values.

The term “reference pattern” is used herein to mean a set of biological attributes that are measured in a normal or diseased biological system under specified experimental conditions. For example, the reference pattern of an allergic asthmatic might include measurements performed on lung exudate via broncho-alveolar lavage, lung function via spirometry, lung tissue via biopsy, or blood via venipuncture at a specified time following a particular chemical mediator or antigen stimulus. Alternatively, the reference pattern of APC behavior might include measurements on cell cultures derived from a normal or diseased human or animal under defined conditions.

### Computer System

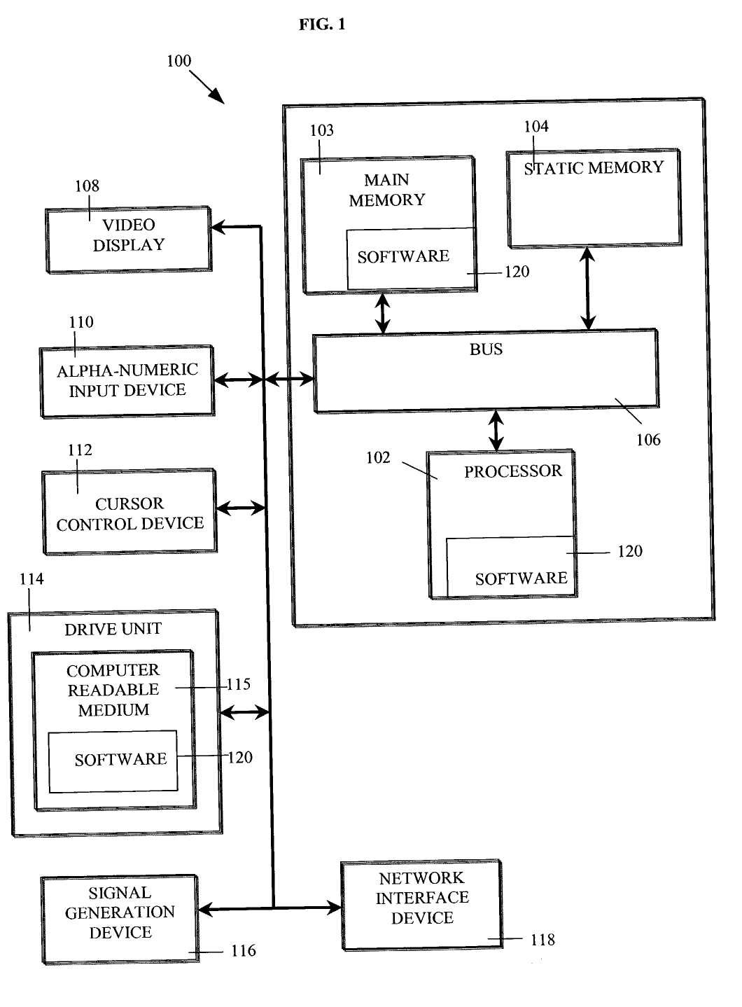

FIG. 1 shows a system block diagram of a computer system within which the methods described above can operate via software code, according to an embodiment of the present invention. The computer system 100 includes a processor 102, a main memory 103 and a static memory 104, which are coupled by bus 106. The computer system 100 can further include a video display unit 108 (e.g., a liquid crystal display (LCD) or cathode ray tube (CRT)) on which a user interface can be displayed. The computer system 100 can also include an alpha-numeric input device 110 (e.g., a keyboard), a cursor control device 112 (e.g., a mouse), a disk drive unit 114, a signal generation device 116 (e.g., a speaker) and a network interface device medium 118. The disk drive unit 114 includes a computer-readable medium 115 on which software 120 can be stored. The software can also reside, completely or partially, within the main memory 103 and/or within the processor 102. The software 120 can also be transmitted or received via the network interface device 118.

The term “computer-readable medium” is used herein to include any medium which is capable of storing or encoding a sequence of instructions or codes for performing the methods described herein and can include, but not limited to, optical and/or magnetic storage devices and/or disks, and carrier wave signals.

### The Computer Model

Suitably, a computer model can be used to implement at least some embodiments of the present invention. The computer model can be used for a variety of purposes. For example, it can enable a researcher to: (1) simulate the dynamics of the biological state associated with an adaptive immune response, (2) visualize key biological pathways for the initiation and maintenance of an adaptive immune response and the feedback within and between these pathways, (3) gain a better understanding of the physiology of an adaptive immune response, (4) explore and test hypotheses about adaptive immune responses, (5) identify and prioritize potential therapeutic targets, (6) identify different types of response and their underlying mechanisms, (7) identify surrogate markers of response types, and (8) organize knowledge and data that relate to the adaptive immune response.

In addition to simulation capabilities, the computer model can include a built-in database of references to the scientific literature on which the model is based. Users can augment this database with additional references or other commentary and can link the information to the relevant component. The computer model can be a multi-user system in which the information can be shared throughout an organization. Thus, the computer model can be a specialized knowledge management system focused on the adaptive immune response.

While the following discussion is in terms of a computer model, one of skill in the art would recognize that the mathematical equations of the model may be analytically or numerically implemented without the assistance of a computer.

Effect Diagram and Summary Diagram

In one embodiment, the computer model can represent various biological components or mechanisms through the use of an Effect Diagram, including a summary diagram and more detailed modules that represent the various biological processes of the biological system being modeled. These modules provide not only a conceptual map of the model, but also represent and encode sets of ordinary differential equations for numerical integration, as discussed more fully below in the section entitled “Mathematical Equations Encoded in the Effect Diagram”.

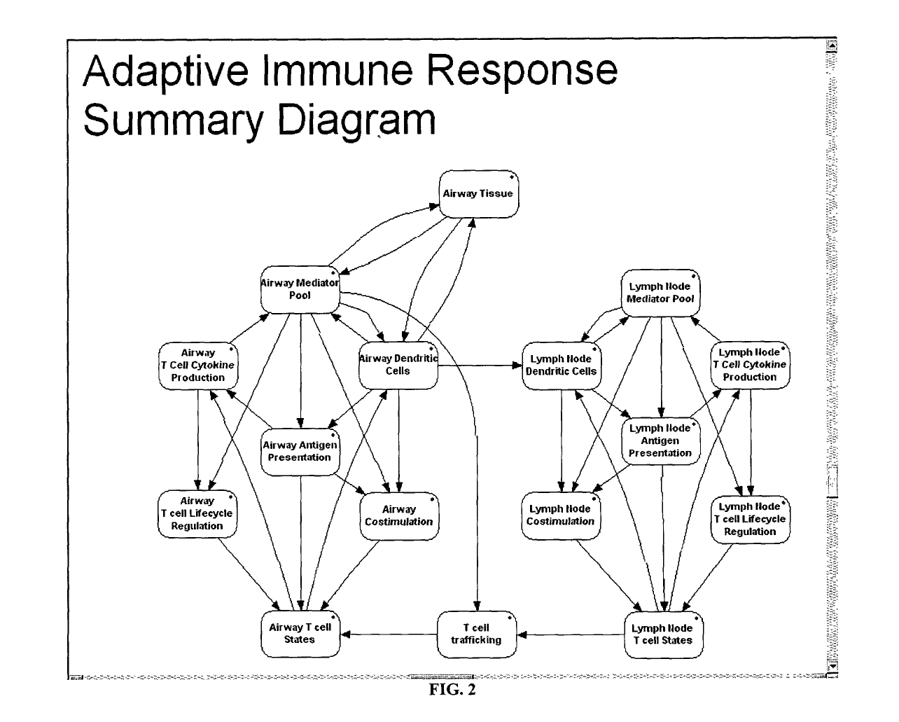

FIG. 2 is a summary diagram that depicts the adaptive immune response components of a model and their interconnectedness, according to an embodiment of the present invention, where the airway is an example of a peripheral tissue. Each squared node shown in FIG. 2 represents a functional module diagram discussed more fully below. The combined functional Effect Diagrams can represent and model the recruitment, phenotypic maturation, antigen processing, death, and departure of APCs in a representative peripheral tissue and lymph node (LN); the recruitment, expansion, differentiation, death, and departure of T lymphocytes in a representative LN and peripheral tissue; and the production of chemical mediators by these cells, subject to the time-varying stimuli of antigen and inflammatory signals from the peripheral tissue.

In one embodiment, antigen presentation can be characterized in the computer model by the availability of APCs, specifically dendritic cells (DCs) over time. Antigen presentation is further characterized by the average antigen density per cell, the average expression of costimulatory molecules (e.g., CD80, CD86) per cell, and DC mediator production. The degree of costimulation accompanying antigen presentation is also determined by the average expression of costimulatory counter-receptors (e.g., CD28) on T lymphocytes being activated. Costimulation effects involving other accessory molecules can be modeled indirectly. For example, CTLA-4 can be modeled indirectly by varying the effective role of CD28.

T lymphocyte population dynamics in the mathematical model are regulated through cell-cell interaction, cytokine production, and cytokine effects. In each representative tissue compartment, cytokine producing cells contribute to a common cytokine pool; different subpopulations of T lymphocytes—described below—respond to cytokine. The nature of the combined T lymphocyte populations and the ambient cytokine milieu vary over time in tandem, each influencing the other.

The particular Effect Diagrams shown below are discussed in reference to particular biological functions (e.g., DC recruitment; DC states, T lymphocyte states). Pages A-1 through A-17 show the complete set of Effect Diagrams included in the present embodiment of the invention.

Mathematical Equations Encoded in the Effect Diagram

As mentioned above, the Effect Diagram is a visual representation of the model equations. This section describes how the diagram encodes a set of ordinary differential equations. Note that although the discussion below regarding state and function nodes refers to biological variables for consistency, the discussion also relates to variables of any appropriate type and need not be limited to just biological variables.

State and Function Nodes

State and function nodes display the names of the variables they represent and their location in the model. Their arrows and modifiers indicate their relation to other nodes within the model. State and function nodes also contain the parameters and equations that are used to compute the values or their variables in simulated experiments. In one embodiment of the computer model, the state and function nodes are generated according to the method described in U.S. Pat. No. 6,051,029 and co-pending application Ser. No. 09/588,855, both of which are entitled “Method of generating a display for a dynamic simulation model utilizing node and link representations,” and both of which are incorporated herein by reference. Further examples of state and function nodes are further discussed below.

[symbol] State nodes, the single-border ovals in the Effect Diagram, represent variables in the system the values of which are determined by the cumulative effects of its inputs over time.

State node values are defined by differential equations. The predefined parameters for a state node include its initial value (Sₒ) and its status. State nodes that have a half-life have the additional parameter of a half-life (h) and are labeled with a half-life [symbol] symbol.

[symbol] Function nodes, the double-border ovals in the Effect Diagram, represent variables in the system the values of which, at any point in time, are determined by inputs at that same point in time.

Function nodes are defined by algebraic functions of their inputs. The predefined parameters for a function node include its initial value (Fₒ) and its status.

Setting the status of a node effects how the value of the node is determined. The status of a state or function node can be

Computed—the value is calculated as a result of its inputs

Specified-Locked—the value is held constant over time

Specified Data—the value varies with time according to predefined data points.

State and function nodes can appear more than once in the Effect Diagram as alias nodes. Alias nodes are indicated by one or more dots, as in the state node illustration above. All nodes are also defined by their position, with respect to arrows and other nodes, as being either source nodes (S) or target nodes (T). Source nodes are located at the tails of arrows, and target nodes are located at the heads of arrows. Nodes can be active or inactive. Active nodes are white. Inactive nodes match the background color of the Effect Diagram.

State Node Equations

The computational status of a state node can be Computed, Specified-Locked, or Specified Data.

$$State Node Computed \frac{ⅆ S}{ⅆ t} = \{ \begin{matrix} sum of arrowterms & when h = 0 \\ \frac{ln 1 / 2}{h} S ( t ) + sum of arrowterms & when h > 0\end{matrix}$$

Where S is the node value, t is time, S(t) is the node value at time, t, and h is the half-life. The three dots at the end of the equation indicate there are additional terms in the equation resulting from any effect arrows leading into it and by any conversion arrows that lead out of it. If h is equal to 0, then the half-life calculation is not performed and dS/dt is determined solely by the arrows attached to the node.

$$State Node Specified \text{-} Locked S ( t ) = {S}_{0} for all t$$

State Node Specified Data S(t) is defined by specified data entered for the state node.

State node values can be limited to a minimum value of zero and a maximum value of one. If limited at zero, S can never be less than zero and the value for S is reset to zero if it goes negative. If limited at one, S cannot be greater than one and is reset to one if it exceeds one.

Function Node Equations

Function node equations are computed by evaluating the specified function of the values of the nodes with arrows pointing into the function node (arguments), plus any object and Effect Diagram parameters used in the function expression. To view the specified function, click the Evaluation tab in the function node Object window.

The Effect Diagram—Arrows

Arrows link source nodes to target nodes and represent the mathematical relationship between the nodes. Arrows can be labeled with circles that indicate the activity of the arrow. A key to the annotations in the circles is located in the upper left corner of each module in the Effect Diagram. If an arrowhead is solid, the effect is positive. If the arrowhead is hollow, the effect is negative.

Arrow Types

[symbol] Effect arrows, the thin arrows on the Effect Diagram, link source state or function nodes to target state nodes. Effect arrows cause changes to target nodes but have no effect on source nodes. They are labeled with circles that indicate the activity of the arrow.

[symbol] Conversion arrows, the thick arrows on the Effect Diagram, represent the way the contents of state nodes are converted into the contents of the attached state nodes. They are labeled with circles that indicate the activity of the arrow. The activity may effect the source node or the target node or both nodes. The conversion can go either way.

[symbol] Argument arrows specify which nodes are input arguments for function nodes. They do not contain parameters or equations and are not labeled with activity circles.

Arrow Characteristics

Effect or conversion arrows can be constant, proportional, or interactive.

[symbol] Arrows that are constant have a break in the arrow shaft. They are used when the rate of change of the target is independent of the values of the source and target nodes.

[symbol] Arrows that are proportional have solid, unbroken shafts and are used when the rate of change is dependent on, or is a function of, the values of the source node.

[symbol] Arrows that are interactive have a loop from the activity circle to the target node. hey indicate that the rate of change of the target is dependent on, or a function of, the value of both the source node and the target node.

Arrow Properties can be displayed in an Object window (not shown). The window may also include tabs for displaying Notes and Arguments associated with the arrow. If Notes are available in the Object window, the arrow is labeled with a red dot (•).

Arrow Equations: Effect Arrows

Proportional Effect Arrow: The rate of change of target tracks source node value.

$$\frac{ⅆ T}{ⅆ t} = C \cdot {S ( t )}^{a} + …$$

Where T is the target node, C is a coefficient, S is the source node, and a is an exponent.

Constant Effect Arrow: The rate of change of the target is constant.

$$\frac{ⅆ T}{ⅆ t} = K + …$$

Where T is the target node and K is a constant.

Interaction Effect Arrow: The rate of change of the target depends on both the source node and target node values.

$$\frac{ⅆ T}{ⅆ t} = C ( {S ( t )}^{a} - {T ( t )}^{b} ) + …$$

Where T is the target node, S is the source node, and a and b are exponents.

This equation can vary depending on the operation selected in the Object window. The operations available are S+T, S−T, S*T, T/S, and S/T.

Arrow Equations: Conversion Arrows

Proportional Conversion Arrow: The rate of change of the target tracks the value of source node.

$$\frac{ⅆ T}{ⅆ t} = C \cdot R \cdot {S ( t )}^{a} + …$$

$$\frac{ⅆ S}{ⅆ t} = - C \cdot {S ( t )}^{a} + …$$

Where T is the target node, S is the source node, C is a coefficient, R is a conversion ratio, and a is an exponent.

Constant Conversion Arrow: The rates of change of target and source are constant such that an increase in target corresponds to a decrease in source.

$$\frac{ⅆ T}{ⅆ t} = K \cdot R + …$$

$$\frac{ⅆ S}{ⅆ t} = - K + …$$

Where T is the target node, S is the source node, K is a constant, and R is a conversion ratio.

Interaction Conversion Arrow: The rates of change of the target and source depend on both source and target node values such that an increase in target corresponds to a decrease in source.

$$\frac{ⅆ T}{ⅆ t} = R \cdot C ( {S ( t )}^{a} - {T ( t )}^{b} ) + …$$

$$\frac{ⅆ S}{ⅆ t} = - C ( {S ( t )}^{a} - {T ( t )}^{b} ) + …$$

Where T is the target node, S is the source node, a and b are exponents, and R is a conversion ratio. This equation can vary depending on the operation selected in the Object window. The operations available are S+T, S−T, S*T, T/S, and S/T.

The Effect Diagram—Modifiers

Modifiers indicate the effects nodes have on the arrows to which they are connected. The type of modification is qualitatively indicated by a symbol in the box. For example, a node can allow [symbol] , block [symbol] , regulate [symbol] , inhibit [symbol] , or stimulate [symbol] an arrow rate.

A key to the modifier annotations is located in the upper left corner of each module.

Modifier Properties can be displayed in the Object Window. The window may also include tabs for displaying the notes, arguments, and specified data associated with the modifier. If notes are available in the Object window, the modifier is labeled with a red dot (•)

Effect Arrow, Modifier Equation:

$$\frac{ⅆ T}{ⅆ t} = M \cdot f ( \frac{u}{N} ) \cdot arrowterm + …$$

Where T is the target node, M is a multiplier constant, N is a normalization constant, f( ) is a function (either linear or specified by a transform curve), and arrowterm is an equation fragment from the attached arrow.

Modifier Effect

By default, conversion arrow modifiers affect both the source and target arrow terms. However, in some cases, a unilateral, modifier is used. Such modifier will affect either a source arrow term or on target arrow term; it does not affect both arrow terms.

Conversion arrow, Source Only Modifier Equation:

$$\frac{ⅆ S}{ⅆ t} = M \cdot f ( \frac{u}{N} ) \cdot arrowterm + other attached arrow terms$$

Conversion arrow, Target Only Modifier Equation:

$$\frac{ⅆ T}{ⅆ t} = M \cdot f ( \frac{u}{N} ) \cdot arrowterm + other attached arrowterms$$

The equation for a source and target modifier uses both the Source Only equation and the Target Only equation.

When multiplicative and additive modifiers are combined, effect is given precedence. For example, if the following modifiers are on an arrow,

a1,a2: Additive, Source and Target

m1,m2: Multiplicative, Source and Target

A1,A2: Additive, Target Only

M1,M2: Multiplicative, Target Only

then the rates are modified by

Target node: (a1+a2+A1+A2)*(m1*m2)*(M1*M2)

Source node: (a1+a2)* (m1*m2)

Embodiments of the Invention

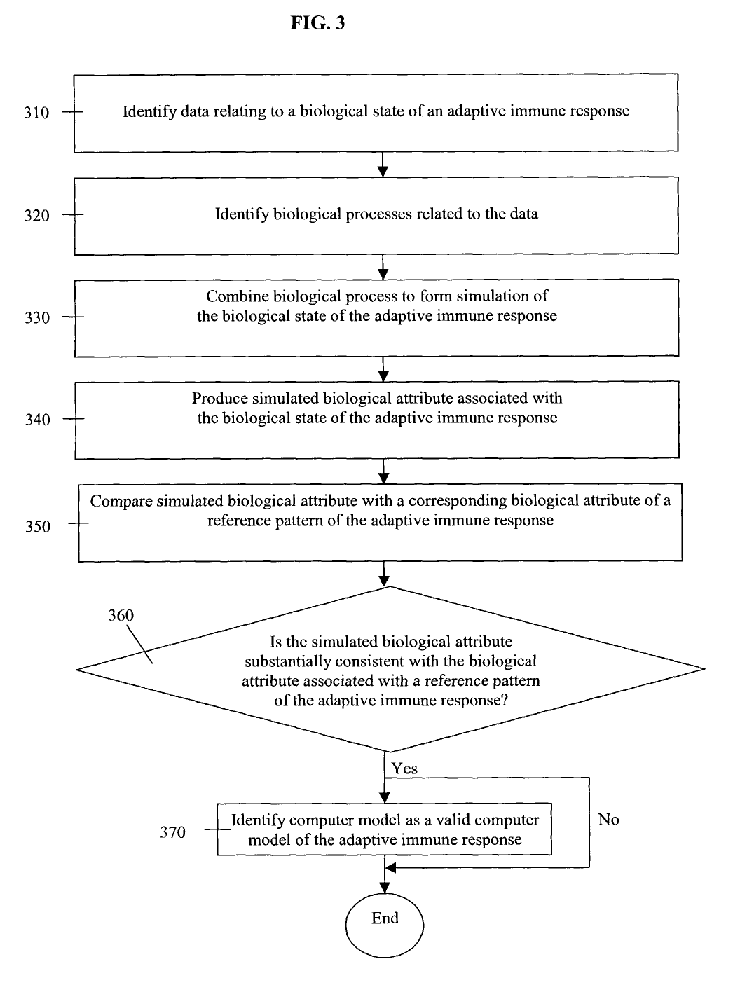

FIG. 3 depicts a flowchart for a method for developing a computer model of an adaptive immune response according to one embodiment of the invention. At step 310, data relating to a biological state of the adaptive immune response is identified. At step 320, biological processes related to the data are identified. These biological processes define at least one portion of the biological state of the adaptive immune response. At step 330, the biological processes are combined to form a simulation of the biological state of the adaptive immune response.

Another embodiment of the invention is a method of developing a computer model of the adaptive immune response wherein the biological state of the adaptive immune response is a biological state of an acute response. In another embodiment of the invention is a method of developing a computer model of the adaptive immune response wherein, the biological state of the adaptive immune response is a biological state of a chronic response. In another embodiment, at least one biological process is associated with a biological variable that is a therapeutic agent. A therapeutic agent of the invention can be, for example, recombinant IL-12, TNF-alpha blockade, steroids, or phosphodiesterase inhibitors.

The method for developing a computer model of an adaptive immune response can further comprise the optional steps of 340, 350, 360, and 370 for validating the computer model, as depicted in FIG. 3. In the validation process, at step 340 a simulated biological attribute associated with the biological state of the adaptive immune response is produced. At step 350, the simulated biological attribute is compared with a corresponding biological attribute in a reference pattern of the adaptive immune response. At steps 360 and 370, the validity of the computer model is identified. At step 360, it is determined whether the simulated biological attribute is substantially consistent with the biological attribute associated with the reference pattern of the adaptive immune response. At step 370, if the simulated biological attribute is substantially consistent with the biological attribute associated with the reference pattern of the adaptive immune response the computer model is identified as a valid computer model of an adaptive immune response.

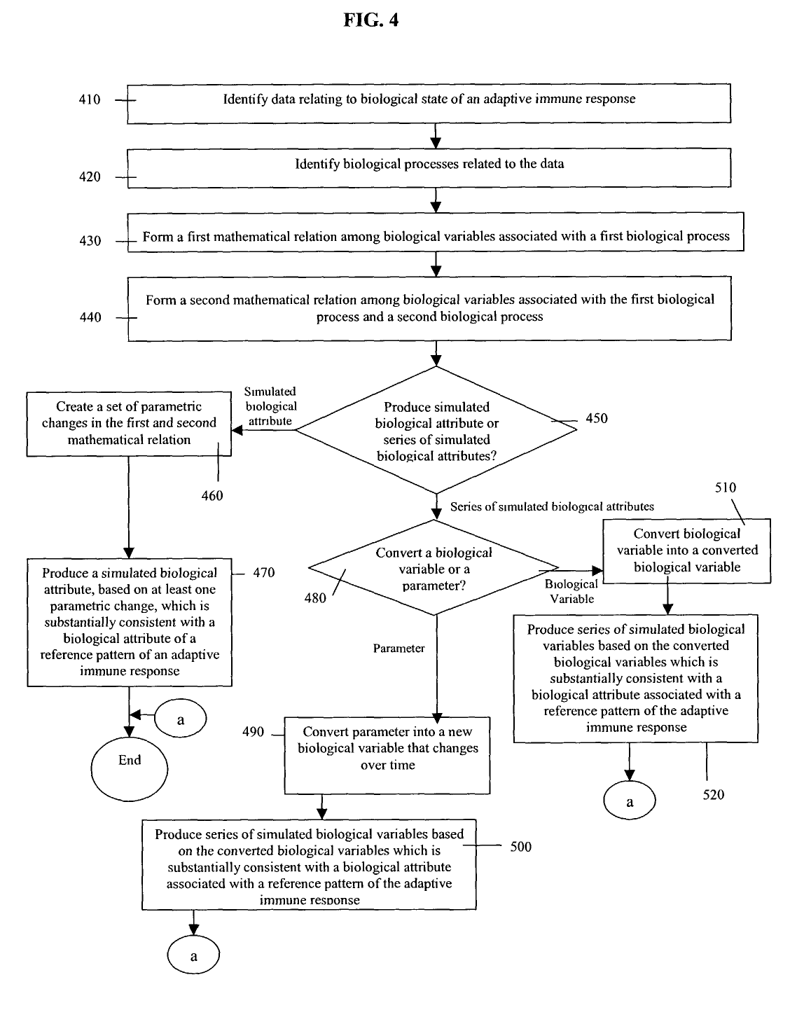

FIG. 4 depicts a flowchart for a method for developing a computer model of an adaptive immune response according to another embodiment of the invention. At step 410, data relating to a biological state of the adaptive immune response is identified. At step 420, biological processes related to the data are identified. These biological processes define at least one portion of the biological state of the adaptive immune response. At step 430, a first mathematical relation among biological variables associated with a first biological process from the biological processes is formed. At step 440, a second mathematical relation among biological variables associated with the first biological process and a second biological process associated with the biological processes is formed.

Steps 450, 460, and 470 can be optionally performed to produce a simulated biological attribute that is substantially consistent with at least one biological attribute associated with a reference pattern of the adaptive immune response. At conditional step 450, a determination is made as to whether a simulated biological attribute or a series of simulated biological attributes is to be produced. If a simulated biological attribute is to be produced, the process continues to step 460. At step 460, a set of parametric changes in the first mathematical relation and the second mathematical relation is created. At step 470, a simulated biological attribute based on at least one parametric change from the set of parametric changes is produced.

Steps 480, 490, 500, 510, and 520 can be optionally performed to obtain a representation of the chronological progression of a diseased adaptive immune response, for example from a healthy state to a disease state. At step 480, a determination is made as to whether a biological variable or a parameter is converted. If a biological variable is to be converted the process proceeds to steps 510, and 520. At step 510, a first biological variable is converted into a converted biological variable the value of which changes over time. This first biological variable is associated with at least one from the first mathematical relation and the second mathematical relation formed in steps 430 and 440. At step 520, a series of simulated biological attributes are produced based on the converted biological variable. The series of simulated biological attributes are substantially consistent with a corresponding biological attribute associated with a reference pattern of the adaptive immune response. The series of simulated biological attributes represent the chronological progression of corresponding biological attributes in the reference pattern of the adaptive immune response. If a parameter is to be converted to obtain a series of simulated biological attributes, the process proceeds to steps 490 and 500. At step 490, a parameter is converted into a new biological variable the value of which changes over time. This parameter is associated with at least one from the first mathematical relation and the second mathematical relation formed in steps 430 and 440. At step 500, a series of simulated biological attributes are produced based on the converted biological variable.

Another embodiment of the present invention is a method for developing a computer model of an adaptive immune response that includes the steps of identifying data related to the biological state of the adaptive immune response; identifying biological processes related to the data, the biological processes defining at least one portion of the biological state of the adaptive immune response; and combining the biological processes to form a simulation of the biological state of the adaptive immune response in the context of a peripheral tissue environment and a lymphoid tissue environment.

Another embodiment of the invention is a method of developing a computer model of the adaptive immune response wherein at least one biological process is associated with recruitment of immune cells into the peripheral tissue environment. In a further embodiment, the method includes immune cells that are blood dendritic cells and blood monocytes. In yet a further embodiment, the method of developing a computer model of an adaptive immune response includes the combining of biological processes so that the peripheral tissue environment is modeled with preferential recruitment of the blood dendritic cells over the blood monocytes.

Another embodiment of the invention is a computer model of a biological state of an adaptive immune response. The computer model comprises code to define biological processes related to the biological state of the adaptive immune response; and code to define mathematical relationships related to interactions among biological variables associated with the biological processes. At least two biological processes from the biological processes are associated with the mathematical relationships. The combination of the code to define the biological processes and the code to define mathematical relationships define a simulation of the biological state of the adaptive immune response in the context of a peripheral tissue environment and a lymphoid tissue environment.

In one embodiment of the computer model of the adaptive immune response at least one biological process is associated with recruitment of immune cells into the peripheral tissue environment. In a further embodiment, the model includes immune cells that are blood dendritic cells and blood monocytes. In yet a further embodiment, the computer model of an adaptive immune response includes the combining of biological processes so that the peripheral tissue environment is modeled with preferential recruitment of the blood dendritic cells over the blood monocytes.

The computer model can further comprise code to define two compartments, wherein one compartment includes biological processes related to a peripheral tissue environment and the second compartment includes biological processes related to a lymphoid tissue environment. Further, the computer model can include a code to define the interaction between these two compartments.

Yet another embodiment of the invention is a computer executable software code that comprises of code to define biological processes related to a biological state of an adaptive immune response including code to define mathematical relations associated with the biological processes. The biological processes defined by the code are associated with the biological state of the adaptive immune response.

The computer executable software can further comprise code to define two compartments, wherein one compartment includes biological processes related to a peripheral tissue environment and the second compartment includes biological processes related to a lymphoid tissue environment. Further, the computer model can include a code to define the interaction between these two compartments.

Additionally, the computer executable software code can comprise code to receive a user selection of a link representation from a set of predefined link representations from a set of predefined link representations, each predefined link representation being uniquely associated with a mathematical relationship. The user-selected link representation is associated with the interrelationship between the first biological variable and the second biological variable, a first link representation from the set of predefined link representations being a representation of the first biological variable having an effect on the second biological variable, a second link representation from the set of predefined link representations being a representation of instances of the first biological variable being converted to instances of the second biological variable.

Another embodiment of the invention is a method for developing a computer model of the biological state of an adaptive immune response, comprising receiving user-selected indications to define biological processes, each biological process being based on data that relates to changes in the adaptive immune response to biological attributes of a reference pattern of adaptive immune response; producing a simulated biological attribute associated with at least one biological attribute of the reference pattern of adaptive immune response; and assessing validity of the computer model based on a comparison between the simulated biological attribute and a corresponding biological attribute associated with the reference pattern of adaptive immune response.

Another embodiment of the invention is a computer model of an adaptive immune response, comprising a computer-readable memory storing codes and a processor coupled to the computer-readable memory, the processor configured to execute the codes. The memory comprises code to define biological processes related to the biological state of the adaptive immune response and code to define mathematical relationships related to interactions among biological variables associated with the biological processes. At least two biological processes defined by the code are associated with the mathematical relationships. The combination of the codes stored in the memory that define the biological processes and the code that defines the mathematical relationships define a simulation of the biological state of the adaptive immune response.

The present invention also includes a method for developing an analytical model of an adaptive immune response. This method includes the steps of identifying data related to the biological state of the adaptive immune response; identifying biological processes related to the data, the biological processes defining at least one portion of the biological state of the adaptive immune response; and combining the biological processes to form an analytical representation of biological state of the adaptive immune response in the context of a peripheral tissue environment and a lymphoid tissue environment.

Another embodiment of the invention is a method of developing an analytical model of the adaptive immune response wherein at least one biological process is associated with recruitment of immune cells into the peripheral tissue environment. In a further embodiment, the method includes immune cells that are blood dendritic cells and blood monocytes. In yet a further embodiment, the method of developing a computer model of an adaptive immune response includes the combining of biological processes so that the peripheral tissue environment is modeled with preferential recruitment of the blood dendritic cells over the blood monocytes.

In one embodiment, in this analytical model, the analytical representation of the biological state of the adaptive immune response can be implemented without the assistance of a computer system.

Another embodiment of the invention is a method for developing a computer model of an antigen-presenting cell, comprising identifying data relating to the physiological regulatory mechanisms of the antigen-presenting cell, the data being associated with at least two from the group of antigen processing, migration, maturation, and mediator production of the antigen-presenting cell and identifying biological processes related to the data, the biological processes defining at least one portion of the role of the antigen-presenting cell in an adaptive immune response. The biological processes are combined to form a simulation of the functioning of the antigen-presenting cell in the adaptive immune response. In one embodiment, in the model the antigen-presenting cell is a dendritic cell. In yet a further embodiment, the antigen-presenting cell is a myeloid dendritic cell. In an additional embodiment, at least one of the biological processes is associated with a differential response to antigen based on the maturational state of the antigen-presenting cell.

### Components of the Mathematical Model

Dendritic Cell (DC) Attributes

DCs are one of several classes of professional antigen-presenting cells, which also include macrophages and B lymphocytes. In one embodiment of the invention, DCs are modeled as the primary APC in a peripheral tissue. DCs continuously traffic between the peripheral tissue and lymphoid tissue and function as sentinels of the immune system by finding and presenting antigens to lymphocytes. The DC mathematical model is designed to represent these biological attributes.

In one embodiment, DC precursors are recruited into a peripheral tissue and differentiate into immature tissue DCs. The total population of DCs, comprised of DCs of all maturational states, is represented by a number of subpopulations which each have a discrete representative maturational state. The flux of DCs from the blood into a peripheral tissue is regulated by local environmental cues. Since the expression of adhesion molecules and chemoattractant receptors is dependent on maturational state, the rates of DC flux are roughly related to the rate of maturation. Once in the tissue, the flux of DCs between these different subpopulations and on to the LNs is regulated by the rate of maturation.

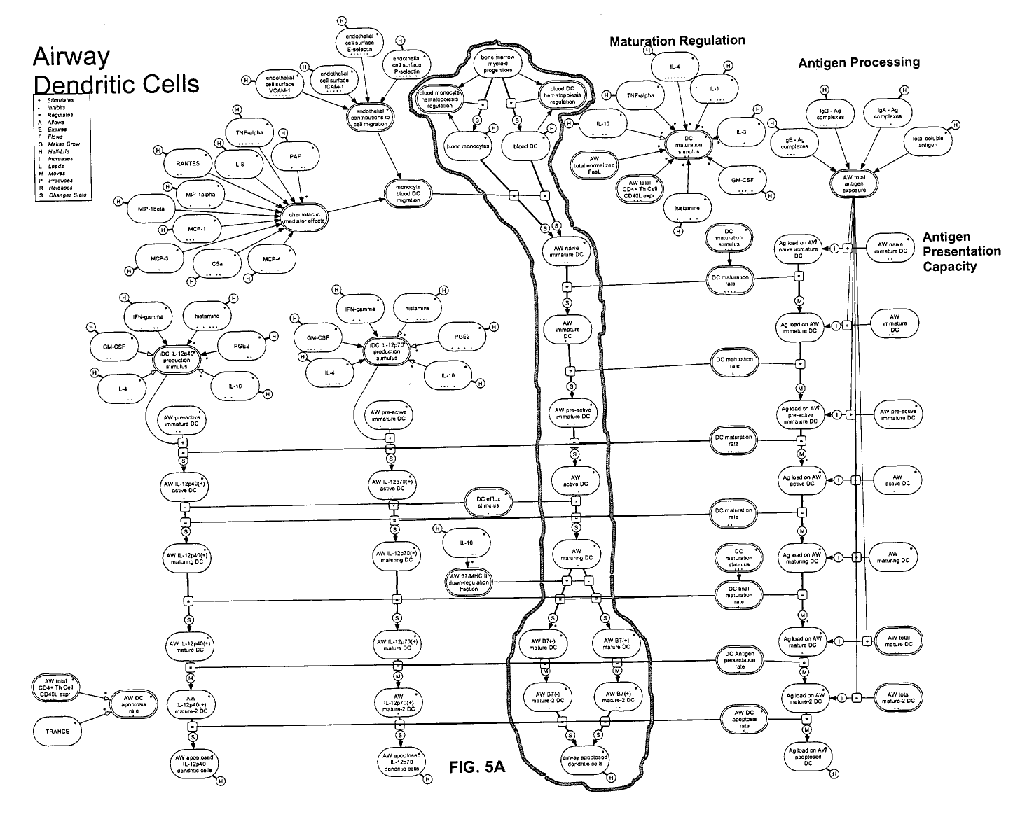

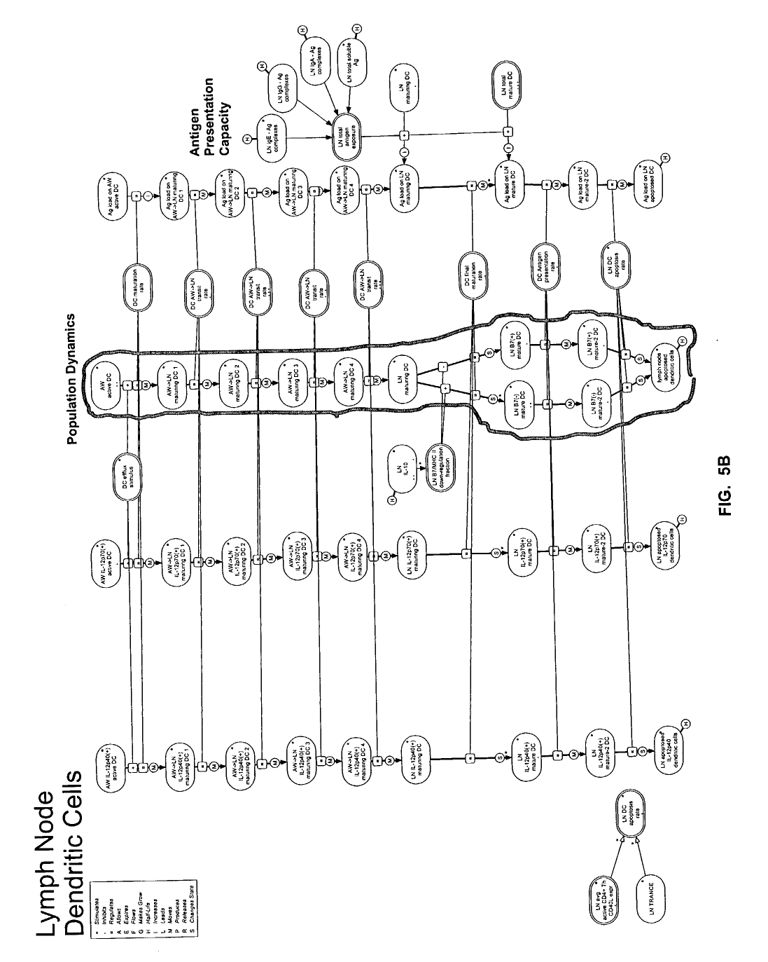

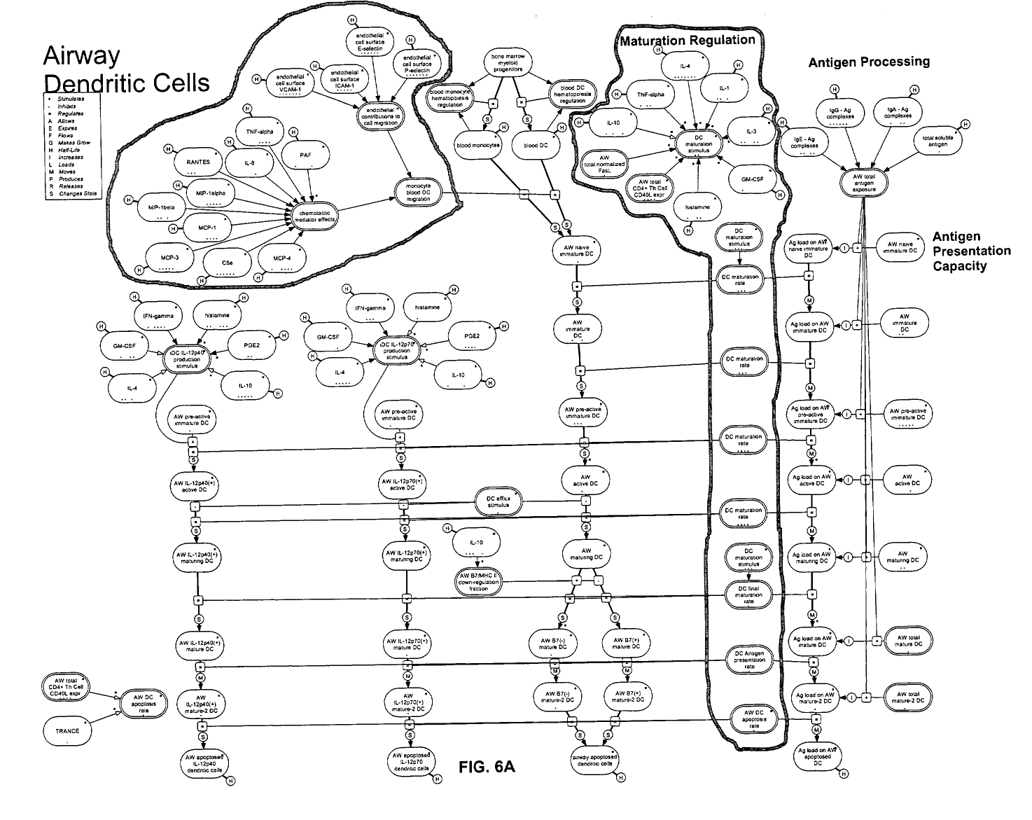

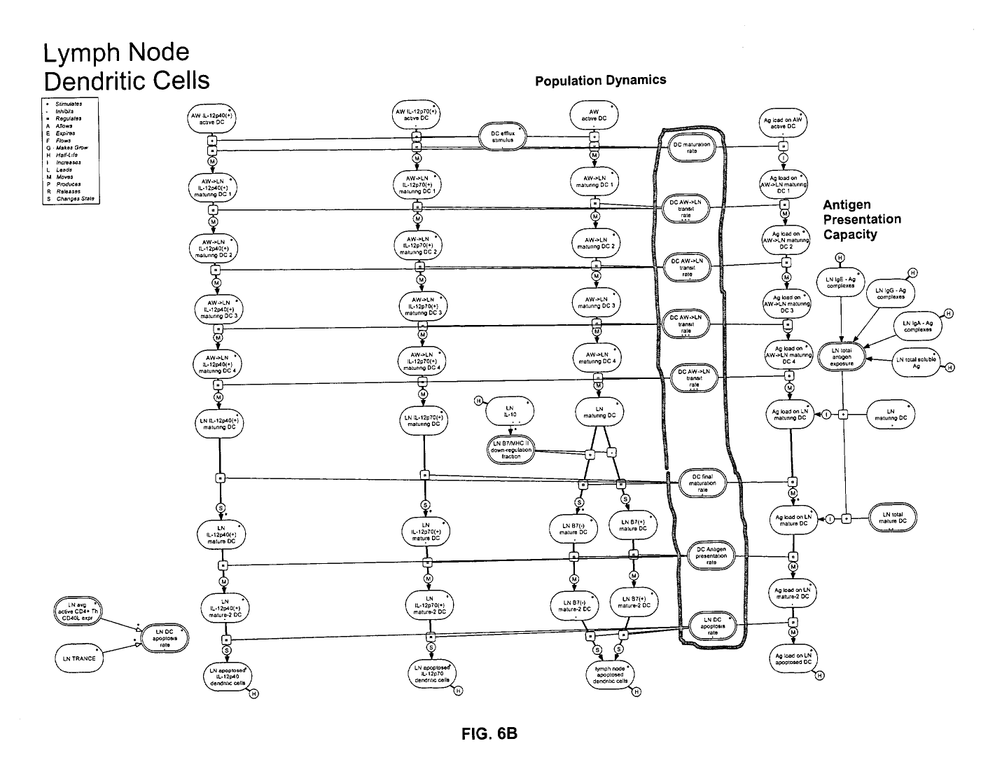

The series of state nodes which represent the DC subpopulations in one embodiment is highlighted in FIG. 5A and FIG. 5B. The interrelationship between nodes represents the flux of DCs from the blood into the tissue and on to the LNs. In general, the maturation process, or the transition between subpopulation states, is characterized by distinct functional and cell surface marker changes, including downregulation of tissue homing chemokine receptors; antigen internalization, processing, and presentation capabilities; upregulation of costimulatory surface molecules (e.g., CD80, CD86); and upregulation of chemokine receptors for lymphoid tissue homing. The rate of DC maturation is modulated by the peripheral tissue microenvironment, for example, through cell-cell interactions (e.g. CD40 ligand-CD40 and Fas ligand-Fas) and through cytokines (e.g., IL-1, IL-3, IL-4, IL-10, TNF-alpha, and GM-CSF). FIG. 6A and FIG. 6B highlight the areas of the DC module in one embodiment that control the rate of DC influx to the peripheral tissue and maturation rate.

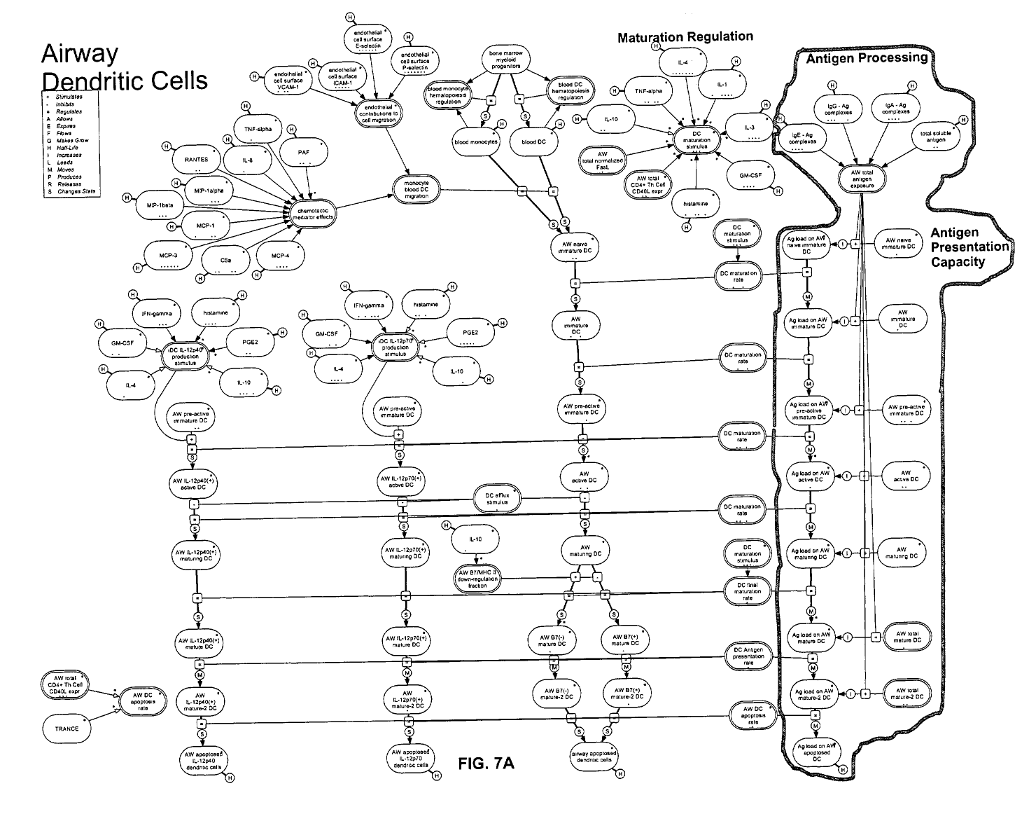

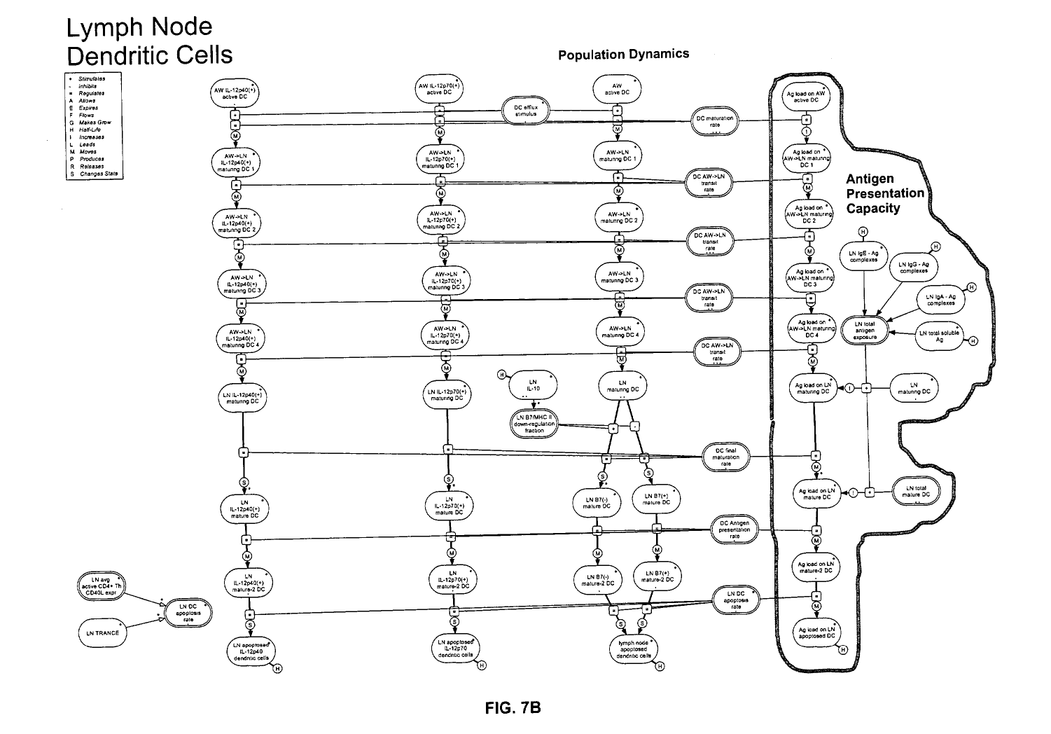

The capability for an individual DC subpopulation to process antigen and activate T lymphocytes is dependent on the average maturational state of that subpopulation. Immature DCs in the peripheral tissue are extremely efficient at internalizing and processing antigen, but are inefficient at stimulating T lymphocyte activation. In contrast, mature DCs are efficient at initiating T lymphocyte activation, but are inefficient at internalizing and processing antigen. The highlighted regions in FIG. 7A and FIG. 7B represent the combined effect of the antigen processing dependence on maturation and the kinetics of antigen availability in the tissue.

Only DCs that have received the appropriate stimuli are capable of activating antigen-specific T lymphocytes. Mature DCs express costimulatory molecules, including CD80 and CD86, and can potently activate T lymphocytes. Expression of costimulatory molecules is limited to mature DCs and is regulated by local environmental cues, such as the presence of IL-10. Mature DCs can present antigen to T lymphocytes in both peripheral tissues and lymphoid tissues. In a lymphoid tissue, DCs can present antigen, express costimulatory molecules, produce mediators that influence T helper lymphocyte differentiation, and be affected by T lymphocyte-DC cognate interactions. The DC ability to present antigen to T cells is dependent on the integrated effects of the kinetics of antigen availability in the tissue, the kinetics of DC maturation, and the ability of DCs to process antigen at discrete points during the maturational process.

DC Cytokine Production

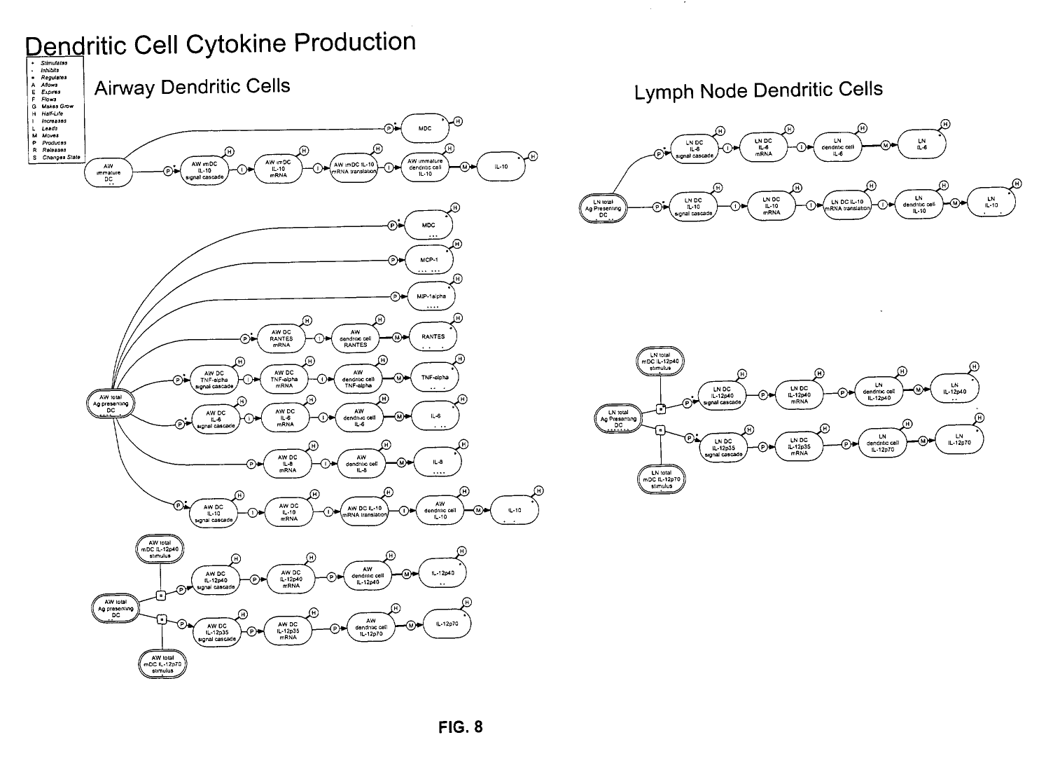

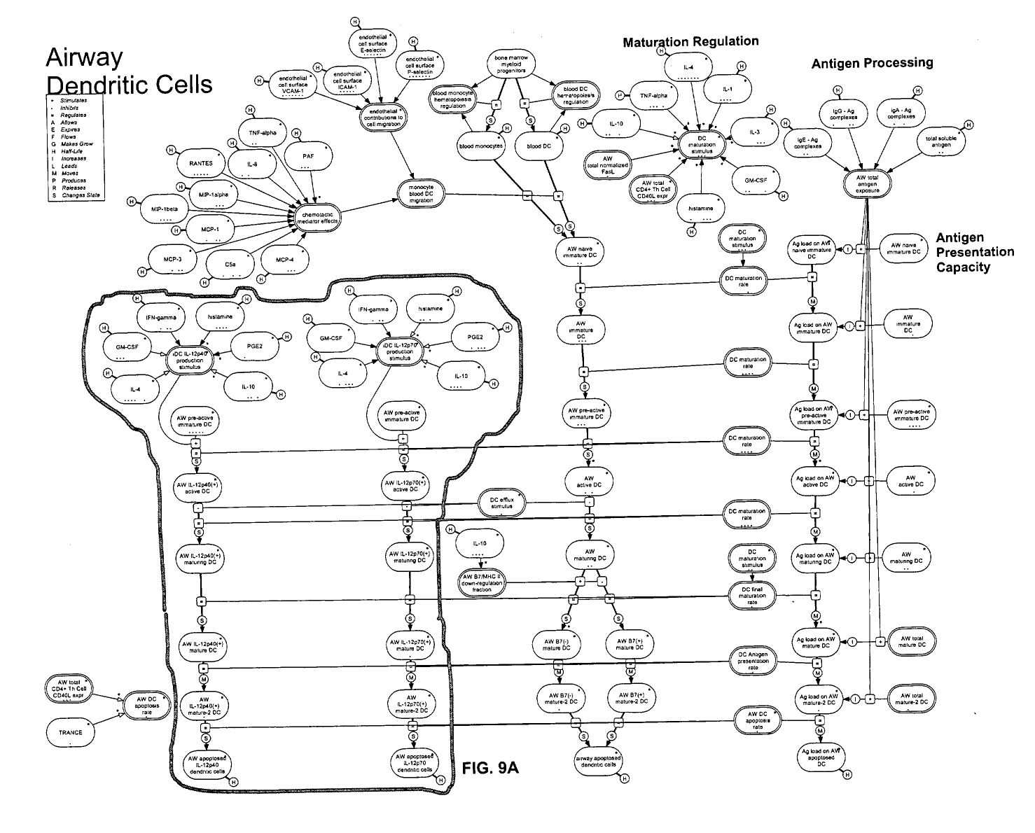

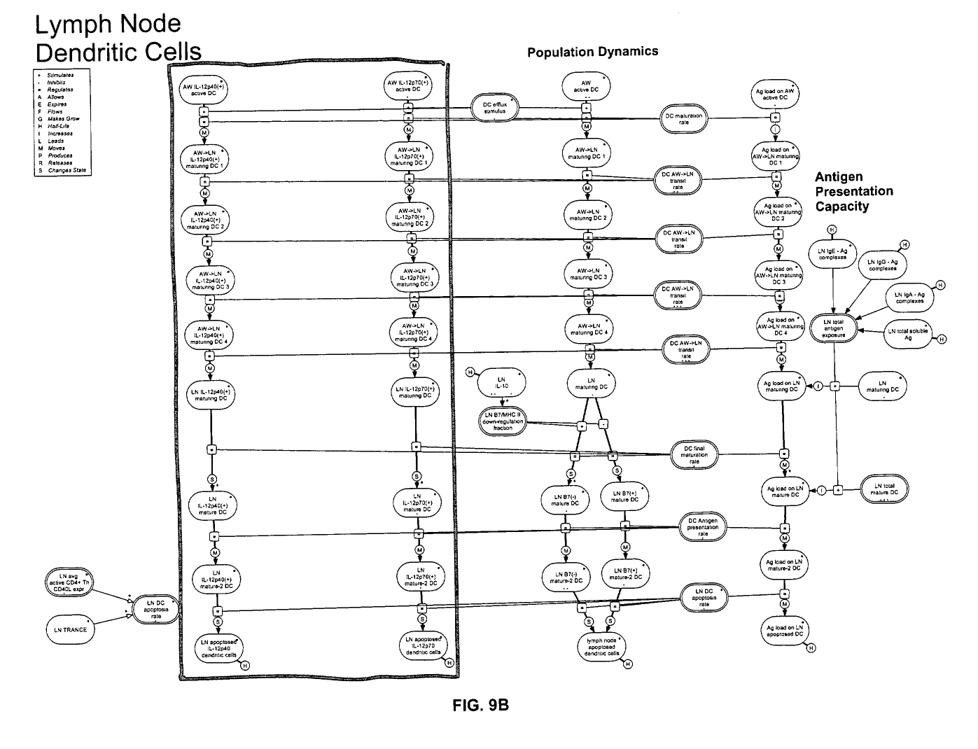

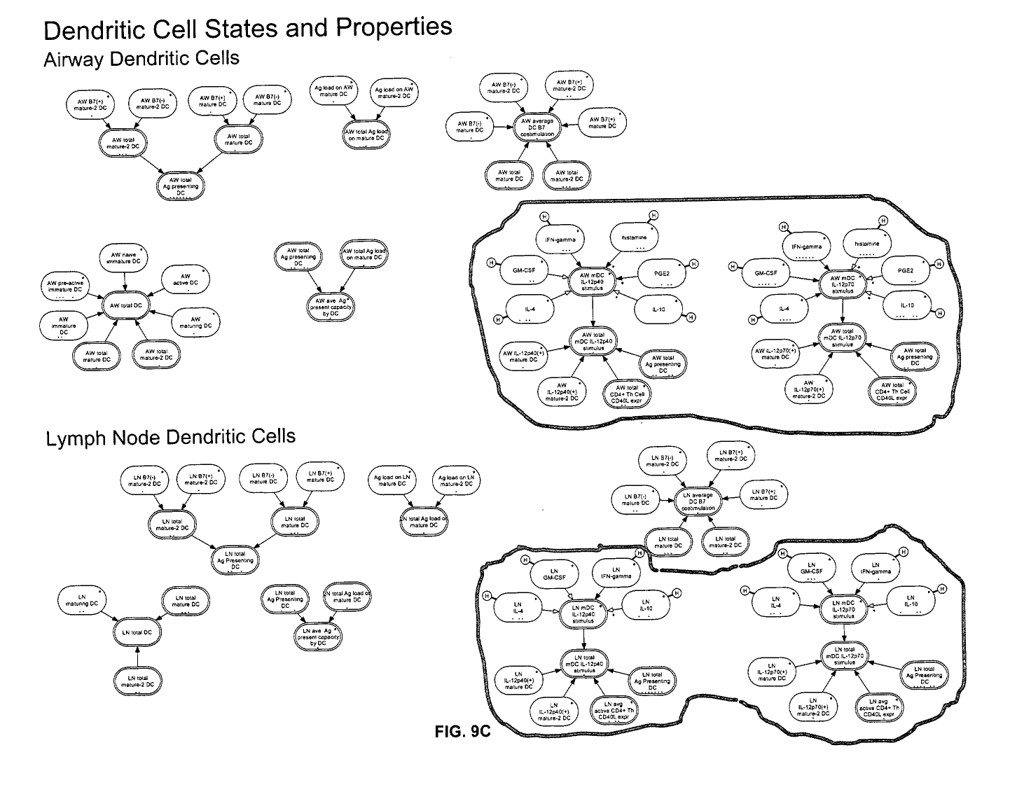

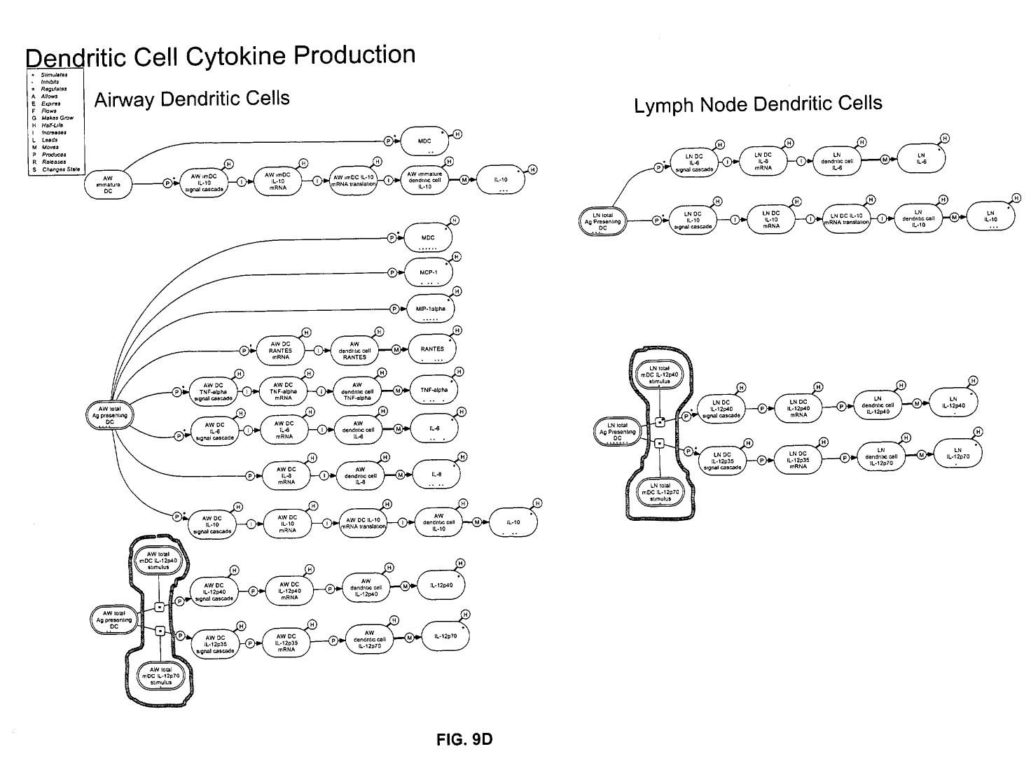

DCs can produce mediators throughout their lifecycle; this production is dependent on maturational state and local environmental cues. Mediators produced by DCs can include, for example, MDC, MCP-1, MIP-1α, RANTES, TNF-α, IL-6, IL-8, IL-10, IL-12p40, and IL-12p70. The nodes in FIG. 8 represent the regulation and production of DC mediators. The activity attributed to IL-12 is dependent on the concentration of the bioactive IL-12p70 heterodimer and the antagonists: IL-12p40 homodimer and IL-12p40 monomer. The production of the possible IL-12 complexes (IL-12p70 heterodimer, IL-12p40 homodimer, or IL-12p40 monomer) is dependent on the integrated exposure to local environmental cues during the DC maturational process. The highlighted regions of FIG. 9A, FIG. 9B, FIG. 9C and FIG. 9D represent this maturational dependence of the IL-12 complexes.

T Lymphocyte States

T lymphocytes and B lymphocytes are the two classes of cells that possess antigen-specific recognition receptors. The activation and subsequent expansion of antigen-specific cells subsequently lead to effector function in the immune response. In one embodiment of the invention, DC activation of CD4+ T helper (Th) lymphocytes and their subsequent expansion and differentiation were modeled.

Presented with the appropriate antigen, in sufficient quantity, and appropriate costimulatory signals, antigen-specific T lymphocytes can be activated to expand and differentiate. The path of differentiation may be regulated by the nature of the antigen stimulus, cytokines, and costimulatory molecules. Differentiated phenotypes have been identified and defined according to their pattern of cytokine production. By these definitions, a Th1 population produces interferon-γ (IFN-γ) and interleukin-2 (IL-2), for example, while a Th2 population produces IL-4 and IL-5. Interestingly, data suggest that even under Th1 or Th2 polarizing conditions, the T lymphocyte population may express multiple cytokines with different kinetics en route to becoming a polarized and largely terminally differentiated population.

With this in mind, in one embodiment, the model can stratify the generic LN T lymphocyte population into subpopulations distinguished by characteristic patterns of cytokine production. Unpolarized, Th1, and Th2 populations exist, but these are subdivided to distinguish, for example, Th2 cells which produce IL-4 from Th2 cells which produce both IL-4 and IL-10. Different patterns of cytokine production by the T lymphocyte population as a whole can be realized by different distributions of cells among these subpopulations.

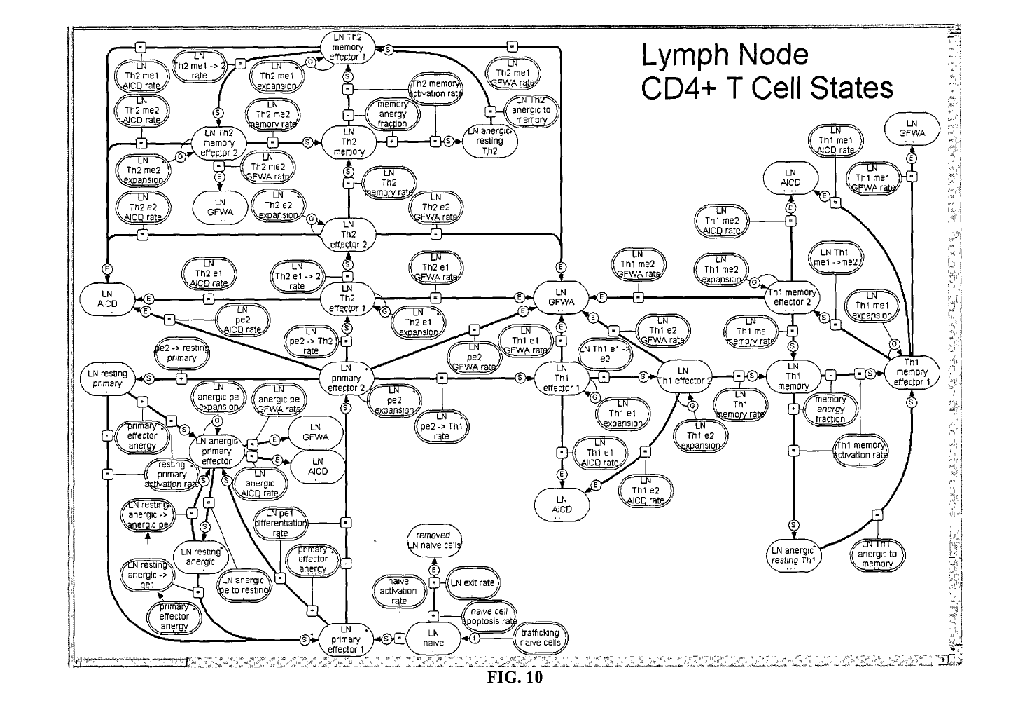

FIG. 10 shows an example of an Effect Diagram that displays a taxonomy of T lymphocyte states, according to an embodiment of the present invention. Naïve cells enter and leave the LNs according to a steady state approximation. They can be made anergic or activated by antigen presentation. Following sufficient antigen stimulation, T lymphocytes produce IL-2 while in the primary effector 1 state (node labeled “LN primary effector 1” in FIG. 10. Cells leaving the subsequent primary effector 2 state (node labeled “LN primary effector 2” in FIG. 10) will assume a Th1 effector phenotype (node labeled “LN Th1 effector” in FIG. 10) or a Th2 effector 1 phenotype (node labeled “LN Th2 effector” in FIG. 10) according to the binding of their receptors for IFN-γ and IL-4. Polarized Th1 memory cells (node labeled “LN Th1 memory” in FIG. 10) and Th2 memory cells (node labeled “LN Th2 memory” in FIG. 10) compete with naïve cells for antigen presented on DCs, and are themselves susceptible to anergy. A fraction of polarized effector T cells also traffic away from the LN, and a fraction of trafficking effector T cells subsequently gain entry to the peripheral tissue.

Subpopulations of T cells are further distinguished by varying susceptibilities to cell death in response to cytokine levels or cell-cell contact. For example, the modeled Th1 effector cells undergo activation-induced cell death (AICD) more rapidly than the modeled Th2 effector cells. The model can include AICD and/or growth factor-withdrawal-induced apoptosis (GFWA).

T Lymphocyte Cytokine Production

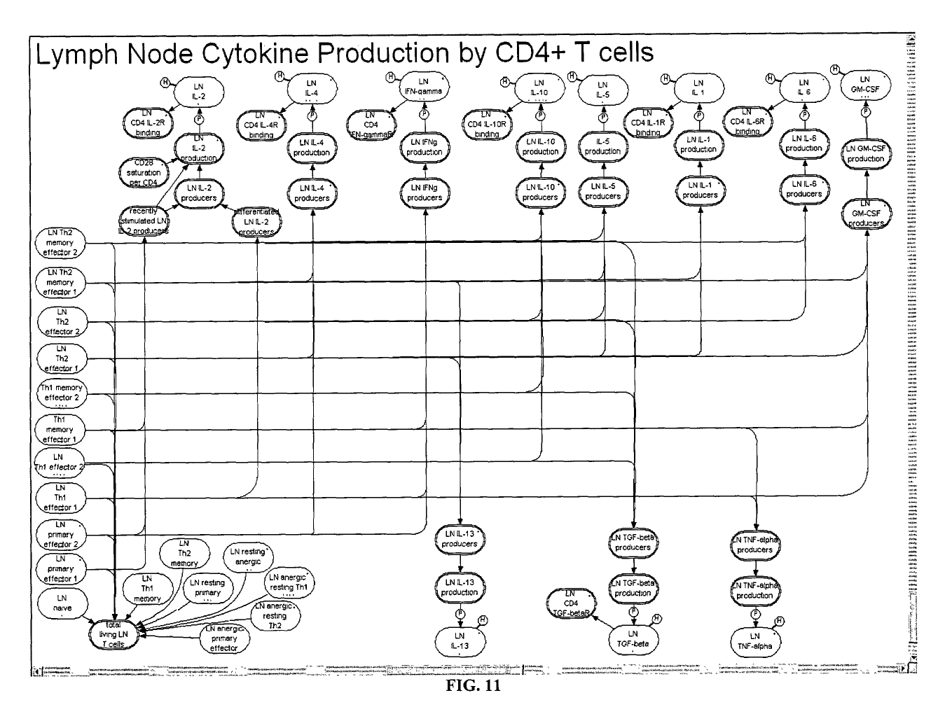

Several cytokines produced by LN T lymphocytes are involved in regulation of T lymphocyte, B lymphocyte, and DC LN populations. These can include, for example, IL-2, IL-4, IL-5, IL-6, IL-10, and IFN-γ. In addition, T lymphocytes that traffic to a peripheral tissue produce cytokines and chemokines involved in the regulation of T lymphocytes and other cell populations (e.g., macrophages, eosinophils) in a peripheral tissue. These can include, for example, IL-4, IL-5, IL-16, IFN-γ, MIP-1β, and 1-309. FIG. 11 shows an example of a portion of an Effect Diagram that calculates T cell cytokine production. Each defined T lymphocyte state produces a particular profile of cytokines at a specified rate. The kinetics of production are modified by the rate at which cells move from one state to the next.

T Lymphocyte Surface Molecule Expression and Ligation

In one embodiment, the modeled T lymphocytes can express several cell surface molecules involved in the regulation of various cell populations. The computer model can accommodate cell surface molecules involved in T lymphocyte regulatory function including costimulation (e.g., CD28), cellular activation (e.g., CD40 ligand), and cell death (e.g., Fas ligand).

T Lymphocyte Regulation of Expansion, Differentiation and Apoptosis

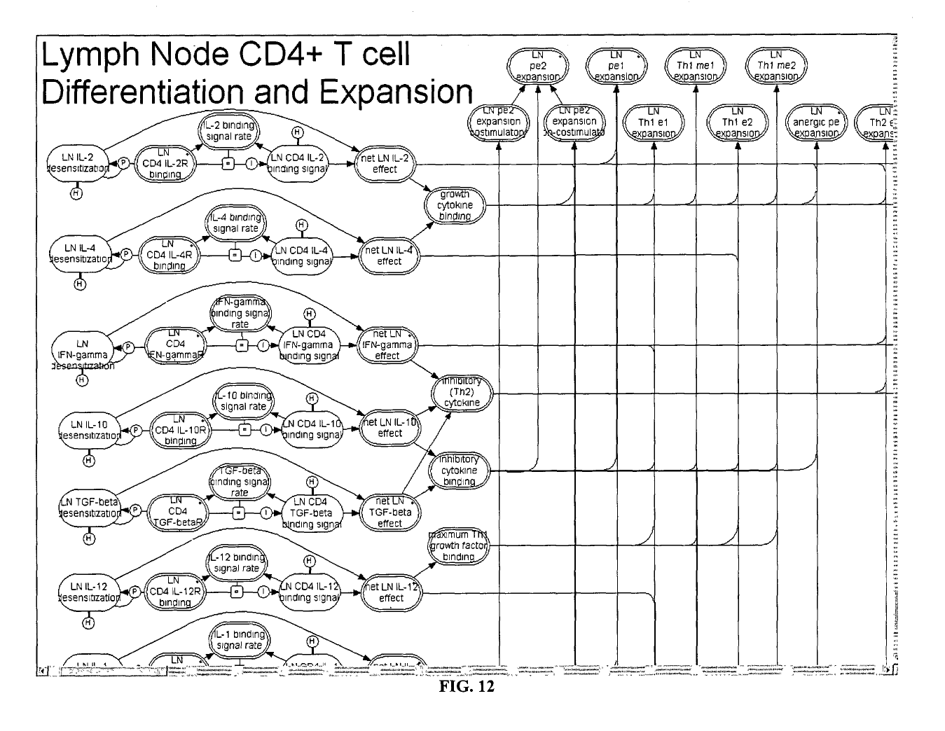

FIG. 12 shows an example of a portion of an Effect Diagram relating cytokine binding and cell-cell interactions to T lymphocyte expansion, differentiation, and death, according to an embodiment of the present invention. T lymphocyte expansion and differentiation are modified by cytokine growth factors, cytokine inhibitory factors, and costimulation. T cell death is modified by insufficient concentration of cytokine growth factors (e.g., IL-2, IL-4) or by activation-induced cell death (AICD). AICD occurs following ligation of death receptors (e.g., Fas) on the activated T cell surface.

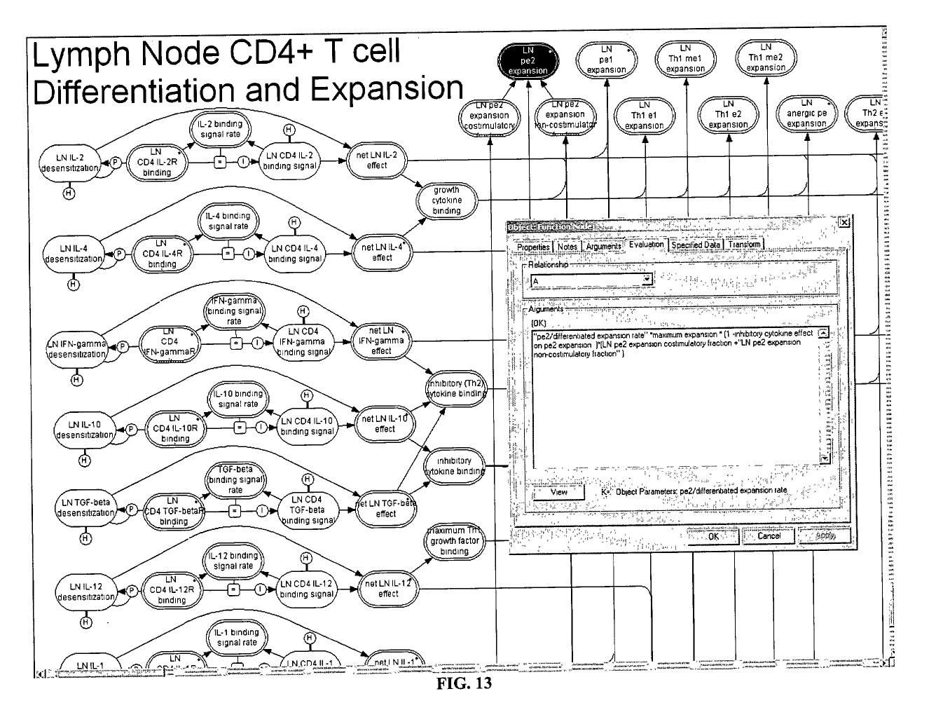

FIG. 13 shows the Effect Diagram of FIG. 12 with an overlay that shows the equation used to calculate LN pe2 expansion function, according to an embodiment of the present invention. As shown in FIG. 13, an equation for this function incorporates cytokine effects, costimulatory molecule effects, cell cycle time, and a rate of movement for cells from the pe2 state to the next state.

T cell Trafficking

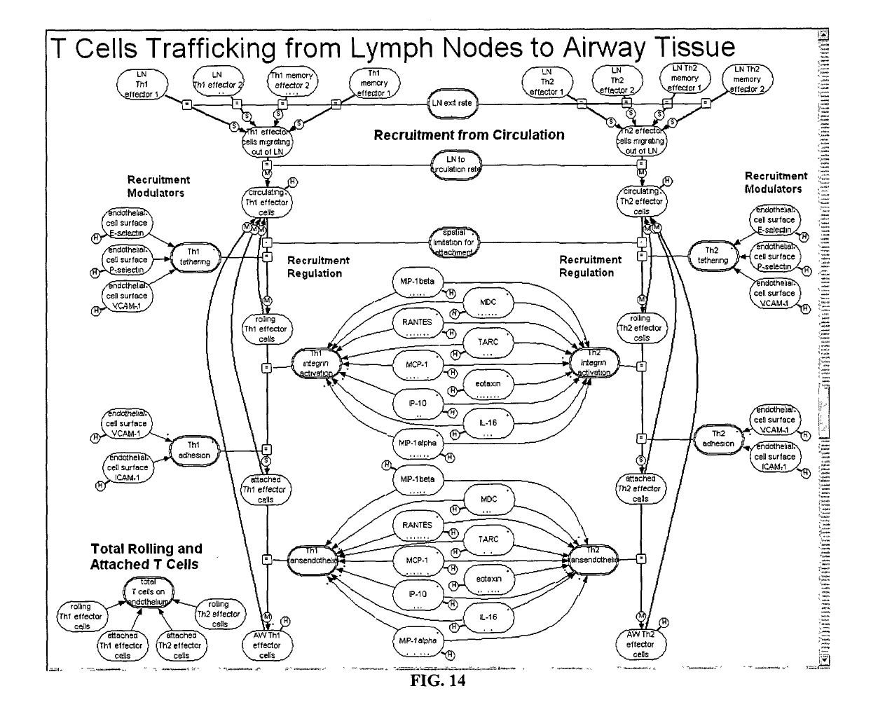

Some fraction of the effector T lymphocyte populations traffic away from the LN. Some of the trafficking T lymphocytes will enter peripheral tissues where they are regulated and may affect other cell populations. T lymphocyte entry to the peripheral tissues is regulated by endothelial expression of several adhesion molecules and by concentration gradients of chemotactic mediators. Cytokines induce endothelial cell expression of several adhesion molecules. These may include P-selectin, E-selectin, vascular cell adhesion molecule-1 (VCAM-1), and intercellular adhesion molecule-1 (ICAM-1). Chemotactic mediators are produced by several cell types in the peripheral tissues and may include, for example, IL-8, IL-16, MIP-1β, thymus and activation regulated chemokine (TARC), and RANTES. Chemotactic mediators may have differential effects on Th1 and Th2 entry to the peripheral tissues as Th1 and Th2 cells express different sets of receptors. FIG. 14 shows an Effect Diagram that depicts Th1 and Th2 cell trafficking as modulated by chemotactic mediators and adhesion molecules.

Emergent Regulation of Th1/Th2 Polarization

It has been shown that Th1/Th2 polarization can be influenced by antigen dose, such that moderate levels of antigen produce a Th1 response while high levels of antigen produce a Th2 response (Hosken et al., J. Exp. Med. 182: 1579-1584, 1995). Similar data exist linking such trends to increased costimulation and intercellular adhesion.

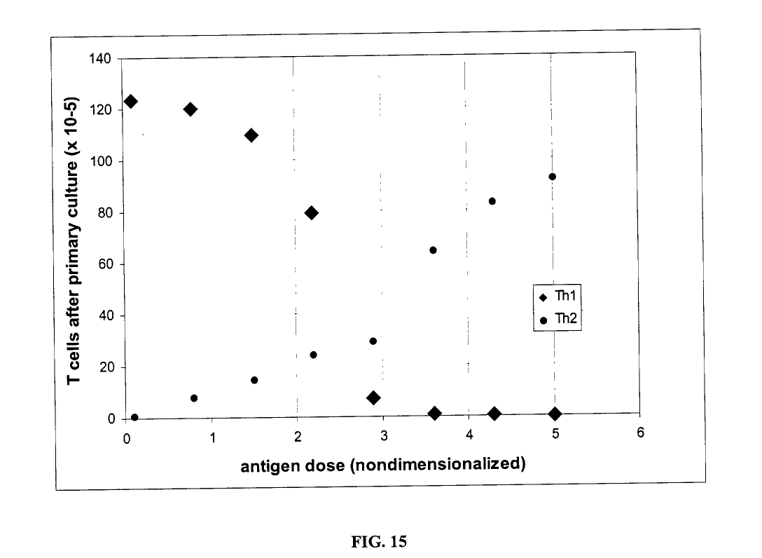

In one embodiment of the present invention, the computer model does not incorporate explicit signaling for naïve cells to produce Th1 or Th2 progeny with varying antigen levels. Nevertheless, this trend is observable as a result of differences in the rates at which Th1 and Th2 cells expand and undergo apoptosis following activation. For example, FIG. 15 illustrates an output of the model showing Th1 and Th2 cells after primary culture as a function of antigen dose, according to an embodiment of the present invention. As FIG. 15 shows, the number of Th1 cells decreases with the antigen dose unlike the number of Th2 cells which increases with the antigen dose.

In another embodiment, the levels of costimulation and—in a rudimentary way—intercellular adhesion can be varied within the computer model to produce similar results. Thus, explicit signaling is not required to observe these shifts in Th1/2 polarization with variations in these factors, and may be reinforced in nature by T cell population dynamics. Published data suggest that varying levels of antigen can produce multiple outcomes in T lymphocyte polarization under different experimental conditions.

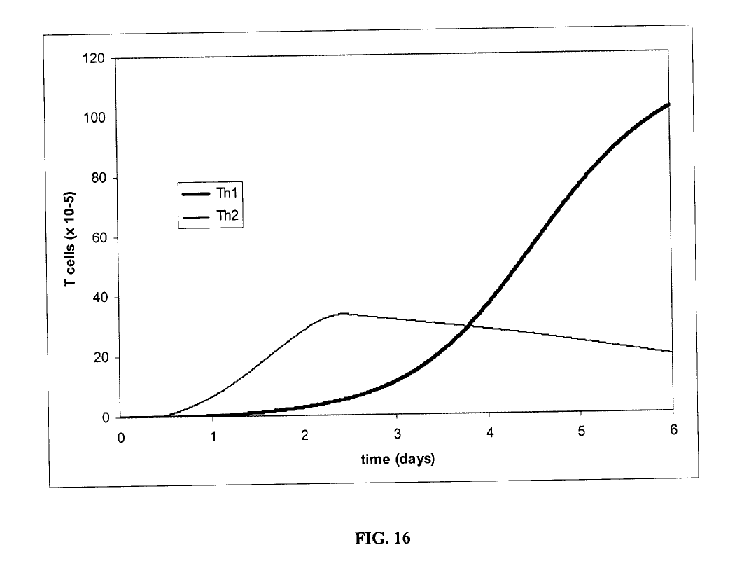

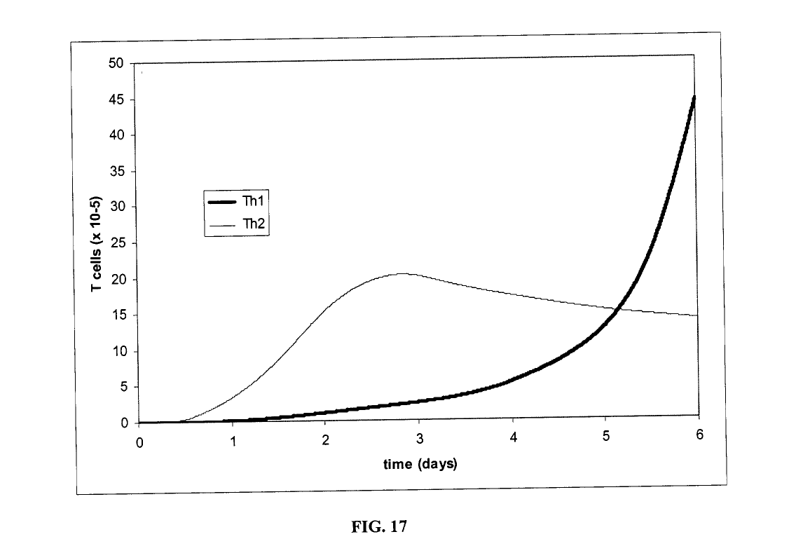

FIG. 16 illustrates an output of the model that depicts evolving numbers of Th1 and Th2 cells during a simulated primary in vitro culture at relatively low antigen dose, according to an embodiment of the present invention. Although the response shown ultimately favors a Th1 phenotype, Th2 cells predominate throughout early population development. As antigen dose is decreased, the phenotypic reversal of such a response occurs later and later in time as shown in FIG. 17. Depending on the point at which cell numbers are measured, an apparent Th2 response will therefore be observed for sufficiently low levels of antigen.

### Example Development of a Model Component: Dendritic Cell Precursor Recruitment to a Peripheral Tissue

The following discussion provides an example of a method by which the components of the above-described mathematical model can be developed. As discussed above, the various elements of the physiologic system are represented by the components shown in the Effect Diagram. These components are denoted by state and function nodes, which, with the arrows and modifiers, represent mathematical relationships that define the elements of the physiologic system. In general, these mathematical relationships are developed with the aid of publicly available or privately generated information on the relevant physiological components. The development of the mathematical relationships underlying the module diagram for DC precursor recruitment into the peripheral tissue will be discussed here as an example.

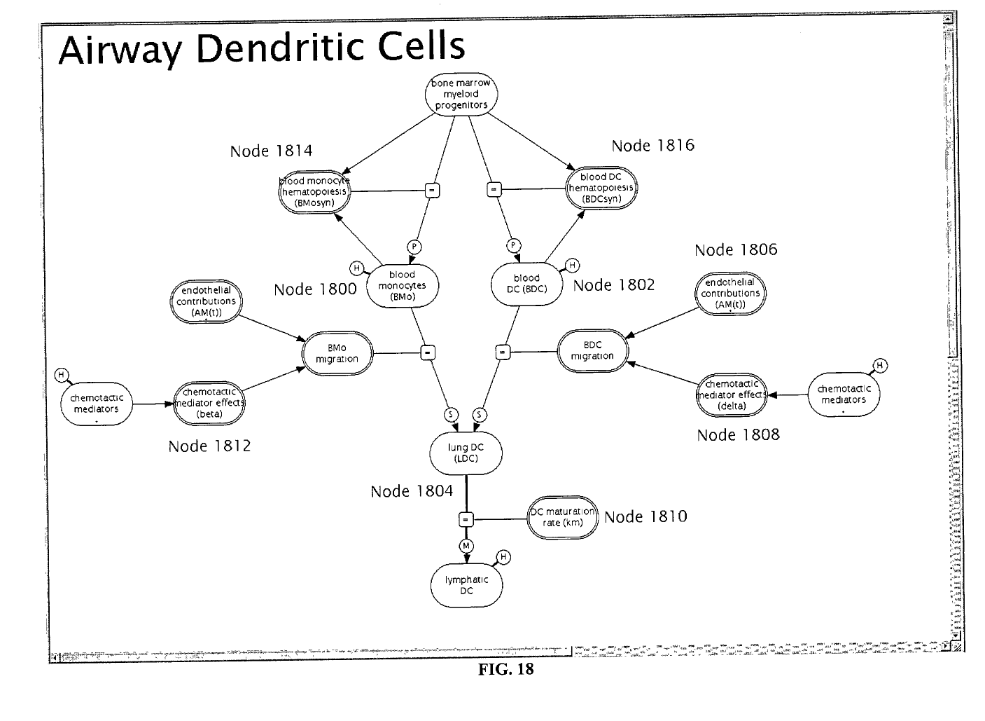

FIG. 18 shows an example of an Effect Diagram for the recruitment of DC precursors into a peripheral tissue, where that peripheral tissue is the lung. For illustration purposes, this Effect Diagram is a simplified version of the Effect Diagram for DC function depicted on page A-2 in Appendix A. The primary focus of this simplified mathematical model is to calculate the contributions of myeloid lung DC precursor populations, which exist in the blood, to the homeostatic and inflammatory response of the tissue DC populations in the human lung. The more detailed mathematical model depicted on page page A-2 in Appendix A also includes the effects of additional mediators on the regulation of DC populations and additional states representing lung DCs.

As FIG. 18 illustrates, maintenance and enhancement of lung DC populations in response to inflammatory stimuli are comprised of the regulated transport of three cell populations: node 1800, blood DC (BDC); node 1802, blood monocytes (BMo); and node 1804, lung DC (LDC). The following discussion relates to deriving the underlying mathematical relationships for these physiological components based on the appropriate publicly available information. Although not discussed herein, the remaining regulation of DC recruitment and population dynamics can be similarly derived from publicly available information.

In the mammalian lung, DCs form an overlying network at the lung surface that has been associated with their role in acquiring and processing antigens at the air-lung surface interface. The total number of LDCs changes dynamically when the lung is exposed to antigen; lung exposure to viral particles, bacteria, or allergen has been shown to temporarily induce up to 3.5-fold increases in the LDC density (McWilliam, A. S. et al., J Exp Med, 179:1331-1336, 1994; Jahnsen, F. L. et al., Thorax, 56:823-826, 2001). To represent these dynamics, the precursor populations for LDCs, the processes governing cellular influx into lung tissue, and the LDC numbers have been modeled.

DC recruitment and associated population dynamics can be quantitatively represented as a set of coupled ordinary differential equations using basic engineering principles such as a conservation of species (Bird, R. B. et al., Transport Phenomena, 2^{nd} Edition (2002), J. Wiley). In general, the relationships for the population dynamics of the three cellular classifications are:

Rate of change of BDC=rate of BDC synthesis−rate of migration of BDC into lungs and other tissue compartments

Rate of change of BMo=rate of BMo synthesis−rate of migration of BMo into lungs and other tissue compartments

Rate of change of LDC=rate of recruitment of BMo+rate of recruitment of BDC−rate of migration of LDC to lymph nodes.

These three cell populations can be normalized to their total population sizes and then the rate of change of the populations can be written as the following equations:

$$\begin{matrix}\frac{ⅆ \overline{BDC}}{ⅆ t} = \frac{{BDC}_{SYN}}{{BDC}_{TOT}} - {k}_{I} \cdot {AM}_{L} ( t ) \cdot δ \cdot \overline{BDC} - {k}_{I} \cdot {AM}_{O} \cdot \overline{BDC} & ( 1 ) \\ \frac{ⅆ \overline{BMo}}{ⅆ t} = \frac{{BMo}_{SYN}}{{BMo}_{TOT}} - {k}_{I} \cdot {AM}_{L} ( t ) \cdot β \cdot \overline{BMo} - {k}_{I} \cdot {AM}_{O}^{′} \cdot \overline{BMo} & ( 2 ) \\ \frac{ⅆ \overline{LDC}}{ⅆ t} = {k}_{I} \cdot {AM}_{L} ( t ) \cdot ( \frac{{BDC}_{TOT}}{{LDC}_{TOT}} \cdot δ \cdot \overline{BDC} + \frac{{BMo}_{TOT}}{{LDC}_{TOT}} \cdot β \cdot \overline{BMo} ) - {k}_{M} \cdot \overline{LDC} & ( 3 )\end{matrix}$$

where

AM_{L}(t)=time−dependent endothelial ligand in lung which facilitates recruitment (sites)

AM_{O}=endothelial ligand in other tissues which facilitates BDC recruitment (sites)

AM′_{O}=endothelial ligand in other tissues which facilitates BMo recruitment (sites)

β=sensitivity of blood monocytes to endothelial ligand expression

δ=sensitivity of blood dendritic cells to endothelial ligand expression

\overline{BDC}=normalized blood dendritic cells (cells/total cells)

\overline{BMo}=normalized blood monocytes (cells/total cells)

\overline{LDC}=normalized lung dendritic cells (cells/total cells)

BDC_{SYN}=synthesis rate of blood dendritic cells (cells/hour)

BDC_{TOT}=total number of blood dendritic cells (total cells)

BMo_{SYN}=synthesis rate of blood monocytes (cells/hour)

BMo_{TOT}=total number of blood monocytes (total cells)

LDC_{TOT}=total number of lung dendritic cells (total cells)

k_{I}=migration influx rate constant (1/(sites hour))

k_{M}=maturation rate constant (1/hour)

Equations (1) and (2) define the dynamic state of BDC and BMo populations as regulated by rates of synthesis and loss to tissue compartments. Equation (3) defines the dynamic state of LDCs as regulated by influx to and efflux from the lung tissue. The first two terms on the right-hand side of the equation (3) imply that once BMo are recruited into the tissue compartment they become LDCs. In one embodiment of the invention, the alternative pathway of monocyte differentiation into other cell types (i.e., macrophages) in the lung has not been represented. The maturation or efflux rate of LDCs from the lung compartment is the third term on the right-hand side of equation (3). This rate of maturation of LDCs represents the migration of mature LDCs into the lymphatic system and, ultimately, to the lymph nodes. This term also includes other pathways for the elimination of LDCs such as apoptosis.

The numerical output of these calculations determines the relative density of LDC, BDC, and BMo at any given time. The values of these variables can then be related to the uptake of antigen, antigen presentation, and other DC activities within the model.

The lung endothelial adhesion molecule expression is dependent on the inflammatory state of the lung. In the short term, the endothelial adhesion molecule expression in other tissues will not be affected by the lung inflammatory response. Thus the kinetics of recruitment to other tissues can be neglected.

The parameters AM′ₒ, k_{I}, and k_{M} can be eliminated using a steady-state approximation for the BDC and BMo populations and equivalent cellular turnover rates. Equations (1)-(3) can then be expressed as:

$$\begin{matrix}\frac{ⅆ \overline{BDC}}{ⅆ t} = \frac{{BDC}_{SYN}}{{BDC}_{TOT}} ( 1 - \frac{δ \cdot {AM}_{L} ( t ) + {AM}_{O}}{δ \cdot {AM}_{L} ( t = 0 ) + {AM}_{O}} \cdot \overline{BDC} ) & ( 4 ) \\ \frac{ⅆ \overline{BMo}}{ⅆ t} = \frac{{BMo}_{SYN}}{{BMo}_{TOT}} ( 1 - \frac{β \cdot {AM}_{L} ( t ) + ( δ - β ) {AM}_{L} ( t = 0 ) + {AM}_{O}}{δ \cdot {AM}_{L} ( t = 0 ) + {AM}_{O}} \cdot \overline{BMo} ) & ( 5 ) \\ \frac{ⅆ \overline{LDC}}{ⅆ t} = \frac{{AM}_{L} ( t )}{δ \cdot {AM}_{L} ( t = 0 ) + {AM}_{O}} \cdot ( \frac{{BDC}_{SYN}}{{LDC}_{TOT}} \cdot δ \cdot \overline{BDC} + \frac{{BMo}_{SYN}}{{LDC}_{TOT}} \cdot β \cdot \overline{BMo} ) - \frac{{AM}_{L} ( t = 0 ) \cdot ( {BDC}_{SYN} \cdot δ + {BMo}_{SYN} \cdot β )}{{LDC}_{TOT} \cdot ( δ \cdot {AM}_{L} ( t = 0 ) + {AM}_{O} )} \cdot \overline{LDC} & ( 6 )\end{matrix}$$

In general, parameter values can be defined based on appropriate measurements in humans. However, in the absence of specific kinetic measurements in humans, data from rodent or other studies were used. The estimates for parameter values for which no experimental data are available can be determined using by the solution of these ordinary differential equations in conjunction with experimental data that represents the appropriate general behaviors to be reproduced. Summed squared error of measurements can be used to determine goodness-of-fit.

Experimentally-Defined Parameters

Experimentally-defined parameters are those whose values could be identified or derived directly from experimental data. These parameters are summarized in Table 1 and discussed below.

TABLE 1
Experimentally-defined parameter values

| Parameter Name | Value |
|---|---|
| BMo_{TOT} | 2.8 × 10⁹ cells |
| BDC_{TOT} | 9.744 × 10⁷ cells |

The total population of blood monocytes (BMo_{TOT}) and blood DC (BDC_{TOT}) were estimated to be 2.8×10⁹ cells and 9.744×10⁷ cells, respectively, based on published estimates of BMo density, BDC density, and assuming that the average human has 5.6 liters of blood (70 kg person, 8% of body weight is blood (Guyton, A. C., Textbook of Medical Physiology, 7^{th} Ed., 1986, W. B. Saunders Company), and density of blood is 1 g/ml). The published estimate of BMo density is 5.0×10⁸ cells/L (Rich, I. N., Monocytes and Macrophages in Primary Hematopoietic Cells (1999) Kluwer Academic Publishers) while that of BDC density is 17.4±5.4×10⁶ cells/L (Upham, J. W. et al., Cytometry, 40:50-59, 2000).

Driving Function which Represents Inflammatory Stimuli

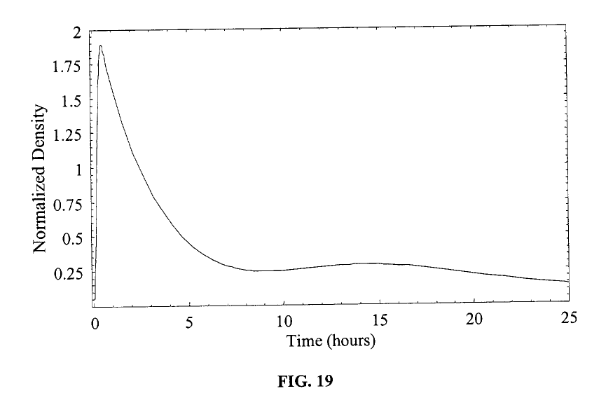

By executing the mathematical model with a driving function, AM_{L}(t), which reflects a dynamic inflammatory stimulus, the equations may be used to determine dynamic changes in recruitment of precursor DC populations into the lung tissue and to determine dynamic increases in the LDC population under inflammatory conditions. The specification of AM_{L}(t) is explained here. Based on the rapid recruitment of the blood cell populations into the lung, P-selectin is the adhesion molecule with expression kinetics most capable of initiating recruitment. P-selectin is stored in Weibel-Palade bodies in endothelial cells and has been observed to increase several-fold quickly on the cell surface, reaching a maximum after 5-10 minutes (e.g., Tedesco, F. et al., J Exp Med, 185:1619-1627, 1997). Disappearance of the surface P-selectin via endocytosis has been observed within 30-60 minutes. In addition, P- and E-selectin can both be transcriptionally induced by inflammatory mediators with similar kinetics and peak approximately 14 hours following stimulation (Vestweber, D. and Blanks, J. E., Physiol Rev, 79:181-213, 1999). To capture these kinetic features, the driving function AML(t) has been estimated to have the time-course shown in FIG. 19. The value of AM_{L}(t) at time equal to zero (AM_{L}(t=0)) was set to 0.05683 sites (Table 2).

TABLE 2
Estimated parameter values for mathematical model

| Parameter Name | Value |
|---|---|
| AM_{L}(t = 0) | 0.05683 sites |

Simulation-Defined Parameters

Simulation-defined parameters are those whose values were selected to yield simulation results that were substantially consistent with experimentally known behaviors of DC precursors in the blood and LDCs. The process of selecting these parameter values involves reproducing experimental protocols in the mathematical model and optimizing parameter values based on the goodness-of-fit of simulation results to experimental results. The simulation-defined parameters are listed in Table 3 and described below.

TABLE 3
Simulation-defined parameter values.

| Parameter Name | Value |
|---|---|
| BMo_{SYN} | 2.526 × 10⁷ cells/hour |
| BDC_{SYN} | 8.792 × 10⁵ cells/hour |
| AM_{O} | 0.02632 sites |
| β | 1 |
| δ | 43.07 |
| LDC_{TOT} | 2.739 × 10⁷ cells |

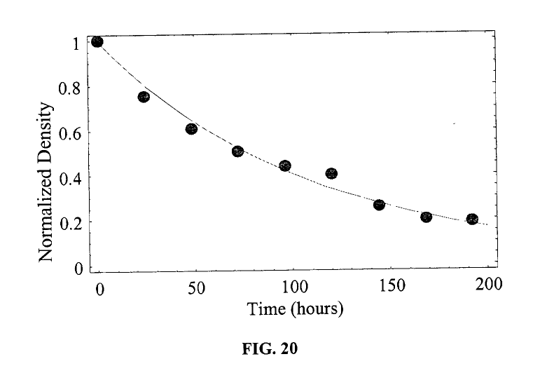

Using data from published radiolabeling studies (Whitelaw, D. M., Blood, 28:455-464, 1966), the turnover rate of BMos was estimated to be 76.8 hours. The experimental data for this radiolabeling study are shown in FIG. 20, and corresponding simulation results using this half-life are also shown. Since both the BMo and BDC come from a common precursor population, the BDC turnover rate was assumed to also be 76.8 hours.

Using a steady-state approximation with equation (1), and this turnover rate, the synthesis rate of blood monocytes (BMo_{SYN}) was calculated to be 2.526×10⁷ cells/hour. The same process with equation (2) gives a calculated synthesis rate of blood DCs (BDCsyn) of 8.792×10⁵ cells/hour.

Values for AM_{O}, β, δ, and LDC_{TOT} were determined from data on steady-state and inflammatory challenge studies of LDCs (Upham, J. W. et al., Am J Respir Crit Care Med, 159:A854, 1999; McWilliam, A. S. et al., J Exp Med, 1994, 179:1331-1336; Holt, P. G. et al., J Immunol, 1994, 153:256-261). The accuracy of the simulation-defined parameters is evaluated against the ability of the mathematical model to reproduce the experimental data. The data used to set the parameter values include the following: Upham et al. reported that BDC and BMo dropped approximately 37% and 5%, respectively, at three hours following antigen challenge. After 24 hours, the BDC level had returned to 78% of the pre-challenge level while the BMo level had returned to normal. McWilliam et al. observed LDC numbers peak at 3.5× baseline values within 1 hour after introduction of an inflammatory stimulus. Holt et al. demonstrated the decline of LDC populations following irradiation of bone marrow.

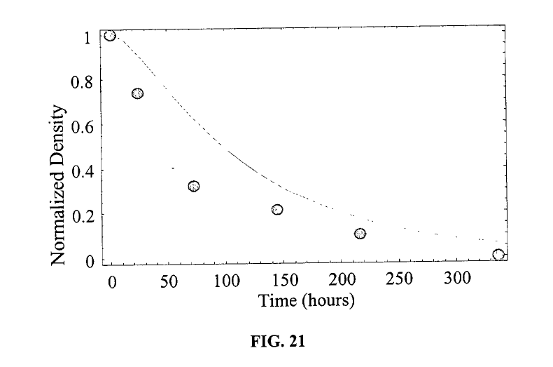

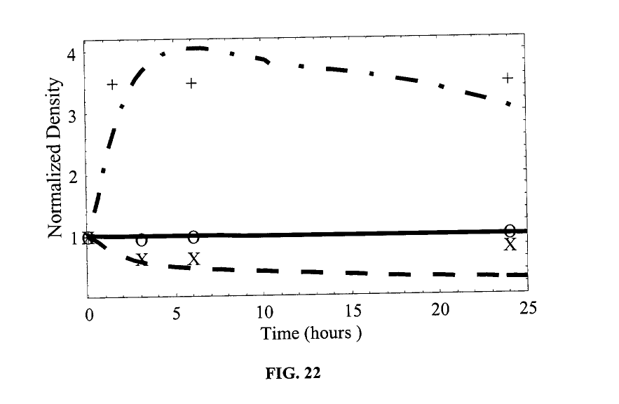

FIG. 21 demonstrates that using the BDC and BMo values defined in Tables 1-3 and equations 4-6 with a simulation protocol that mimics the experimental protocol of Holt et al., the LDC values are in agreement with the experimental findings of Holt et al. In FIG. 22, the simulation results for both the blood and tissue populations are shown to be substantially consistent with the experimental data of Upham et al. and McWilliam et al. The results are also substantially consistent with other measurements of the dynamic response of the LDC populations to various experimental conditions (Ingulli, E. et al., J Exp Med, 1997, 185:2133-2141; Lambrecht, B. N. et al., Am J Respir Cell Mol Biol, 1999, 20:1165-1174; Stumbles, P. A. et al., J Immunol, 2001, 167:228-234).

Useful Outcomes of Mathematical Modeling

Mathematical modeling of DC recruitment as just explained has generated insightful observations on this area of biology. Upham et al. demonstrated that at 3 hours following antigen challenge, BDC density dropped by 37% while BMo density dropped by 5%. In reproducing these experimental data with the model, it was also found that BDC account for 20% of the post-challenge tissue DC population. So, while BDC comprise only about 3.4% of the total DC precursors in the blood (i.e., BDC plus BMo), they comprise a much larger percentage of the lung tissue population. The functional consequences of preferential BDC recruitment may be profound. BDC and BMo are functionally distinct in their antigen processing and presentation abilities (Caux, C. et al., Blood, 90:1458-1470, 1997; Garrett, W. S. et al., Cell, 102:325-334, 2000; Sallusto, F. and Lanzavecchia, A., J Exp Med, 179:1109-1118, 1994; Yang, D. et al., J Immunol, 165:2694-2702, 2000). Hence, preferential recruitment of one cell type over the other may dramatically influence antigen presentation to T lymphocytes.

An additional useful observation is that the kinetics of DC precursor and tissue DC population responses following antigen challenge suggest that P-selectin is the most likely among the known adhesion molecules to mediate recruitment because it can be rapidly mobilized to the endothelial cell surface upon stimulation (Tedesco, F. et al., J Exp Med, 185:1619-1627, 1997).

As this example model of DC precursor recruitment into a peripheral tissue generally illustrates, the components of the Effect Diagram, denoted by state and function nodes and the arrows and modifiers linking them, represent mathematical relationships that define the elements of the physiologic system. These mathematical relationships can be developed with the aid of appropriate publicly available information on the relevant physiological components. In other words, the Effect Diagram indicates the types of mathematical relationships that are represented in the model. The publicly available information can be put into a form that matches the structure of the Effect Diagram. In this way, the structure of the model can be developed.

### Simulation of Biological Attributes of an Adaptive Immune Response

The following discussion describes the nature of the biological attributes that can be obtained by numerical or analytical integration of the mathematical model. It further elucidates changes that may be made to the model to obtain simulated biological attributes that correspond to qualitatively or quantitatively different adaptive immune responses.

The mathematical model is equipped with a set of baseline parameters selected to represent a particular type of adaptive immune response. In one embodiment, the baseline parameters are selected such that the simulated biological attributes are substantially consistent with the biological attributes of an established immune response to an allergic stimulus. The parameters of the model can be changed to represent varying manifestations of the response including for example, acute responses to a bolus exposure of antigen, or low level chronic responses to low levels of antigen, or quiescent response to the absence of antigen.

The model can also be changed parametrically to represent different contributions of the involved biological processes to the biological state. Changing the contributions to the biological processes will yield different simulated biological attributes and enable exploration of how parameter changes affect outcomes. For example, changing the appropriate model parameters such that DC bioactive IL-12 production is enhanced favors Th1 polarizing conditions and enables investigation of the subsequent changes imposed on T lymphocyte populations.

When integrated with other components to generate a normal or diseased physiological system (e.g., allergic asthma), the model can represent the contribution of the immune response to the state of the system or to the progression between disease states of different severity. The model can also represent the different stages of immune responses, i.e., primary and secondary exposure to a given antigen, as well as exposure to varying levels of antigen. One means of generating this variation in the model can involve replacing one or more biological variables, formerly fixed at a particular value, with one or more biological variables that evolve over time and depend on some previously included or new biological processes. For example, in one embodiment, the APCs are exposed to a chronic low level of antigenic stimuli. The low level of antigen exposure results in some basal level of inflammation in the peripheral tissue and maintains a chronic low level immune response. Altering the immune response, to include for example, acute responses, might involve replacing a fixed parameter (e.g., antigen level) with a direct function of time, an algebraic function of other biological variables (i.e., a biological process), or via a dynamical systems equation such an ordinary differential equation. Alternatively, changing the immune response might involve adding new processes such as exposure to viral or bacterial pathogens, influx and dynamics of NK cells and neutrophils, and allowing APC behavior to be modulated by these processes.

In one embodiment, the previously fixed values that specify allergen exposure of a peripheral lung tissue by a mild allergic asthmatic, are replaced by a direct function of time or by a function of other biological variables to represent the effect of seasonal changes in allergic stimuli to the pathology of allergic asthma. The model depiction of acute exacerbations in a chronically inflamed lung can be used to study, for example, the role of the adaptive immune response in acute exacerbations as well as chronic inflammation and approaches to alter the character of the adaptive immune response through therapeutic changes to the APCs themselves or the peripheral tissue environment.

### Model Calibration

Model calibration refers to the estimation of parameter values based on quantitative and qualitative experimental observations that correspond to biological variables, biological processes and/or biological states represented in the mathematical model. Parameter values are estimated from both in vitro and in vivo data from the literature in the model calibration process. With parameter values estimated in this way, the modules of the mathematical model show behavior substantially consistent with experimental studies that focus on specific aspects of the adaptive immune response. For example, the model can reproduce the timing and numbers of cells involved in DC recruitment into peripheral tissue, and the response of different T lymphocyte populations to APCs with varying antigen loads. As shown in the previous section, “Example Development of a Model Component: Dendritic Cell Precursor Recruitment to a Peripheral Tissue”, the recruitment of DCs has been shown to match both blood and peripheral tissue data. Published data specifying the appropriate behavior of the system overall can be used to calibrate remaining degrees of freedom in the computer model.

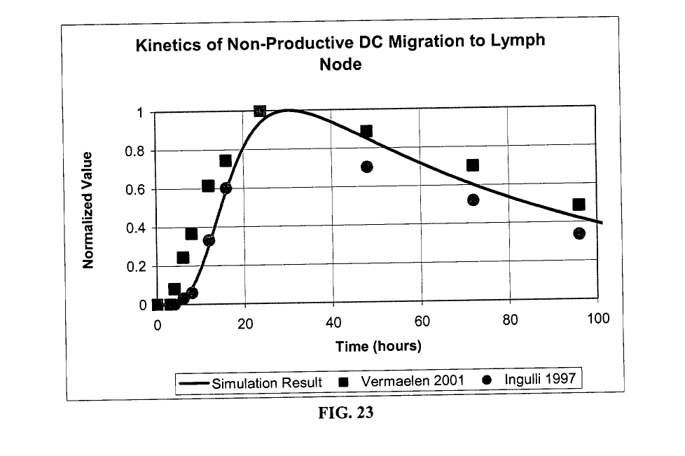

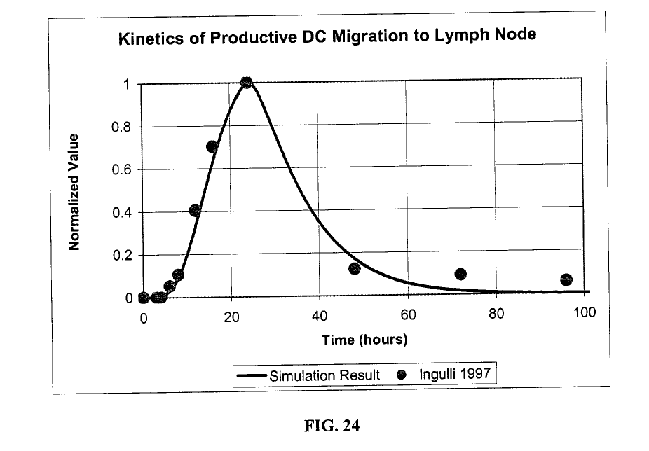

Examples of the calibration of the migration kinetics of DCs into the lymph node are shown in FIG. 23 and FIG. 24. As can be seen in the FIG. 23, the simulation result is substantially consistent with experimental measurements reported by two independent groups in the published literature (Ingulli et al., J Exp Med, 185: 2133-2141, 1997; Vermaelen et al., J Exp Med, 193: 51-60, 2001) in the context of non-productive interactions with CD4+ T lymphocytes. In contrast, productive interactions of DCs with CD4+ T cells result in slightly different kinetics, as seen in FIG. 24. Productive interactions induce activation of the CD4+ T lymphocytes, which in turn increases the apoptosis of the antigen-presenting DCs. The simulation result is consistent with published experimental results (Ingulli et al., J Exp Med, 185: 2133-2141, 1997) and is shown in FIG. 24.

The stimulation of T lymphocytes by APCs also reproduces the experimental data reported in the scientific literature. In Table 4, the simulation results are compared to a study by London et al. (London et al., J Immunol, 164:265-272, 2000) where the cytokine production response of various T lymphocyte populations to antigen and costimulation was measured. The model parameters were selected to be consistent with the experimental protocol described by London et al. in which both memory (mem) and naïve T lymphocyte populations were stimulated with two levels of antigen (Ag) and costimulatory molecules expressed by APCs. The cytokine production kinetics were measured (Table 4), and in some instances several cytokines were not observed above the detection limit (n.d.). The simulation results demonstrate that both naïve and memory populations in the mathematical model respond appropriately in terms of the relative cytokine amount and timing to different levels of antigen. The general behavioral differences between naïve and memory T lymphocyte responses are also consistent with the understanding that memory lymphocytes require less antigen for stimulation as demonstrated by the data of London et al.

TABLE 4

| | | Simulation Results | | Results from London | |
|---|---|---|---|---|---|
| | | Normalize | | et al. 2000 | |
| | T Cell phenotype and | d peak | Time of | Normalized | Time of |
| Cytokine | stimulation | value | peak (h) | peak value | peak (h) |
| IL-2 | Naïve, low Ag | <0.001 | | n.d. | |
| IL-2 | Naïve, high Ag | 0.053 | 62 | 0.04 | 60 |
| IL-2 | Mem, low Ag | 0.122 | 58 | 0.07 | 60 |
| IL-2 | Mem, high Ag | 0.157 | 56 | 0.16 | 60 |
| IL-4 | Naïve (high and low) | <0.001 | | n.d. | |
| IL-4 | Mem, low Ag | 0.007 | 60 | 0.005 | 60 |
| IL-4 | Mem, high Ag | 0.008 | 58 | 0.008 | 84 |
| IFN-gamma | Naïve (high and low) | <0.001 | | n.d. | |
| IFN-gamma | Mem, low Ag | 0.015 | 64 | 0.018 | 60 |
| IFN-gamma | Mem, high Ag | 0.018 | 70 | 0.018 | 84 |

### Initialization of the Mathematical Equations and Numerical Solution of the Computer Model

Since the Effect Diagram defines a set of ordinary differential equations as described above, once the initial values of the biological variables are specified, along with the values for the model parameters, the equations can be solved numerically by a computer using standard algorithms. See, for example, William H. Press et al. Numerical Recipes in C: The Art of Scientific Computing, 2nd edition (January 1993) Cambridge Univ. Press. As illustrated above in the Example Development of a Model Component: Dendritic Cell Precursor Recruitment to a Peripheral Tissue section, one can derive equations, obtain initial conditions, and estimate parameter values from the public literature. Likewise, other initial conditions and parameter values can be estimated for different conditions and can be used to simulate the chronological progression of the biological state.

In one embodiment, the computer executable software code numerically solves the mathematical equations of the model under simulated experimental conditions. For example, one could simulate an in vitro experiment by specifying the duration of the experiment and the following initial conditions for the biological variables: DC density, the DC state(s), the T lymphocyte density, the T lymphocyte population states (e.g., naïve CD4+ T lymphocytes, resting Th1 lymphocytes, resting Th2 lymphocytes), the amount of antigen, and the cytokine environment (e.g., endogenous or exogenous IL-12, IFN-gamma, IL-4). The numerical solution would include the values for all the experimentally measured cell populations and mediator levels (e.g., number of IL-2 expressing Th1 lymphocytes) at the times they were measured in the laboratory. In addition, the numerical solution could generate the complete chronological progression of all biological variables in the model over the course of the experiment.

Furthermore, the computer executable software code can facilitate visualization and manipulation of the model equations and their associated parameters to simulate different patients subject to a variety of stimuli. See, e.g., U.S. Pat. No. 6,078,739, entitled “Managing objects and parameter values associated with the objects within a simulation model,” and U.S. Pat. No. 6,069,629, entitled “Method of providing access to object parameters within a simulation model” the disclosures of which are incorporated herein by reference.

In one embodiment the invention can be used to model therapeutic agents such as steroids, β-agonists, or leukotriene antagonists. Thus, the model can be used to rapidly test hypotheses and investigate potential drug targets or therapeutic strategies. For example, a therapy can be modeled in a static manner by modifying the Parameter Set of the appropriate tissue(s) to represent the affect of the treatment on that tissue(s). Alternatively, therapeutic treatments can be modeled in a dynamic manner by allowing the user to specify the delivery of a treatment(s), for example, in a time-varying (and/or periodic) manner. To do this, the computer model has the ability to include pharmacokinetic representations of various modulatory classes of treatment (e.g., anti-cytokine antibodies, adjuvant-like mediators, steroids) and how these treatments can interact with the various cell types in a dynamic manner. Further, when the model is integrated with a disease model, there is an ability to include pharmacokinetic representations of various therapeutic classes (e.g., anti-cytokine antibodies, altered forms of antigen, adjuvant-like mediators, steroids) and evaluate how these therapeutics interact with the elements of peripheral and lymphoid tissue to generate a clinical outcome.

### Validation of the Model

The behavior of the model is validated by comparing simulated biological processes to reference patterns of those biological processes. Validation in this manner can be used to validate both the computer model and the analytical model embodiments of the invention. For example, one method of validating the behavior of the computer model is validated by comparing simulated biological attributes of the model to reference patterns of individual components of the adaptive immune response or to reference patterns of the entire adaptive immune response. An alternate method is to link the mathematical model of the adaptive immune response to a model of another biological system that interacts with the adaptive immune response, for example a model of an allergic asthmatic lung, a model of intestinal bowel disease (IBD), or a model of Schistomsoma mansoni infection, and compare simulations of the combined model to reference patterns for the combined system.

In one embodiment, validation of the model of the adaptive immune response is performed by linking it to a model of a peripheral tissue whose function depends on the adaptive immune response, and comparing simulation results for the linked model to reference patterns of the peripheral tissue biological function. The model of the adaptive immune response could also be integrated with any number of other components to represent a healthy or diseased physiological system. Validation of the model would be done by comparing simulated results of the linked model to reference patterns of the components, which could be tissues or entire organisms that are dependent on the adaptive immune response.

To use linking of the adaptive immune response to a peripheral tissue model for validation, the following process can be used. The method of validation can involve having a model with the attributes of a peripheral tissue of interest in a particular biological state. Specific characteristics of cell types, cellular abnormalities, physiological abnormalities, and the chemical mediator environment of the peripheral tissue can be modeled appropriately. In particular, the peripheral tissue modeling is necessary to reflect the impact of peripheral tissue constituents on cells of the adaptive immune system and conversely, the impact of immune cells on peripheral tissue constituents. In one embodiment, the adaptive immune response model can modified to reflect the nature of APCs and lymphocytes that are associated with specific biological state of the selected peripheral tissue. The interactions of the immune cells with each other and with cells or chemical mediators of the peripheral tissue result in simulated biological attributes that can be compared to experimentally observable reference patterns. Methods for validation of computer models are described in an application entitled “Apparatus and Methods for Validating a Computer Model,” filed on May 16, 2002, application No. 10/151,581 which is incorporated herein by reference.

The adaptive immune response is implicated in many diseases including allergic asthma, rheumatoid arthritis, inflammatory bowel disease, cancer, and all infectious diseases. As an example of validation by linking with another model, the adaptive immune response model can be validated through incorporation in a disease model of allergic asthma. Under chronic conditions, the adaptive immune response model can, for example, provide the proper stimuli to produce known reference patterns in asthmatic patients including elevated IgE levels, partial degranulation of mast cells, airway hyperresponsiveness, and elevated levels of cytokines associated with a Th2 adaptive immune response. The model can also reproduce appropriate reference patterns of patient responses to bolus doses of antigen, including compromised airway function and elevated cell and chemical mediator levels in the airways.

Consistency with module-specific reference patterns measured in in vivo and in vitro studies, as well as reference patterns of clinical outcomes when incorporated within a full disease model, provides validation for the computer model. The mathematical model of an adaptive immune response can be considered a valid model if the simulated biological attribute obtained is substantially consistent with a corresponding biological attribute obtained from a cellular or whole animal model of an adaptive immune response. As the understanding of the adaptive immune response, and the diseases associated with the adaptive immune response, evolve in the art, the responses against which the model is validated can be modified.

While various embodiments of the invention have been described above, it should be understood that they have been presented by way of example only, and not limitation. Thus, the breadth and scope of the present invention should not be limited by any of the above-described embodiments, but should be defined only in accordance with the following claims and their equivalents.

The previous description of the embodiments is provided to enable any person skilled in the art to make or use the invention. While the invention has been particularly shown and described with reference to embodiments thereof, it will be understood by those skilled in the art that various changes in form and details may be made therein without departing from the spirit and scope of the invention. For example, although a certain embodiment of a computer system is described above, other embodiments are possible. Such computer system embodiments can be, for example, a networked or distributed computer system.

[image]

[image]

[image]

[image]

[image]

[image]

[image]

[image]

[image]

[image]

[image]

[image]

[image]

[image]

[image]

[image]

[image]

What is claimed is:

1. A method for developing a computer model of an adaptive immune response, comprising:
identifying data relating to a biological state of the adaptive immune response;
identifying a plurality of biological processes related to the data, the plurality of biological processes defining at least one portion of the biological state of the adaptive immune response; and
combining the plurality of biological processes to form a simulation of the biological state of the adaptive immune response.

2. The method of claim 1, wherein the biological state of the adaptive immune response is a biological state of an acute response.

3. The method of claim 1, wherein the biological state of the adaptive immune response is a biological state of a chronic response.

4. The method of claim 1, wherein at least one biological process from the plurality of biological processes is associated with a biological variable that is a therapeutic agent.

5. The method of claim 1, further comprising:
producing a simulated biological attribute associated with the biological state of the adaptive immune response;
comparing the simulated biological attribute with a corresponding biological attribute in a reference pattern of the adaptive immune response; and
identifying the computer model as a valid computer model of the adaptive immune response if the simulated biological attribute is substantially consistent with the biological attribute associated with the reference pattern of the adaptive immune response.

6. The method of claim 1, wherein the combining the plurality of biological processes includes:
forming a first mathematical relation among biological variables associated with a first biological process from the plurality of biological processes; and
forming a second mathematical relation among biological variables associated with the first biological process and biological variables associated with a second biological process from the plurality of biological processes.

7. The method of claim 6, further comprising:
creating a set of parametric changes in the first mathematical relation and the second mathematical relation; and
producing a simulated biological attribute based on at least one parametric change from the set of parametric changes, the simulated biological attribute being substantially consistent with at least one biological attribute associated with a reference pattern of the adaptive immune response.

8. The method of claim 6, further comprising:
converting a first biological variable into a converted biological variable a value of which changes over time, the first biological variable being associated with at least one from the first mathematical relation and the second mathematical relation; and
producing a series of simulated biological attributes based on the converted biological variable, the series of simulated biological attributes being substantially consistent with a corresponding biological attribute associated with a reference pattern of the adaptive immune response, the series of simulated biological attributes representing the chronological progression of the corresponding biological attribute in the reference pattern of the adaptive immune response.

9. The method of claim 6, further comprising:
converting a parameter into a new biological variable a value of which changes over time, the parameter being associated with at least one from the first mathematical relation and the second mathematical relation; and
producing a series of simulated biological attributes based on the new biological variable, the series of simulated biological attributes being substantially consistent with a biological attribute associated with a reference pattern of an adaptive immune response, the series of simulated biological attributes representing the chronological progression of corresponding biological attributes in the reference pattern of the adaptive immune response.

10. A method for developing a computer model of an adaptive immune response, comprising:
identifying data relating to a biological state of the adaptive immune response;
identifying a plurality of biological processes related to the data, the plurality of biological processes defining at least one portion of the biological state of the adaptive immune response; and
combining the plurality of biological processes to form a simulation of the biological state of the adaptive immune response in the context of a peripheral tissue environment and a lymphoid tissue environment.

11. The method of claim 10, wherein at least one biological process from the plurality of biological processes is associated with recruitment of immune cells into the peripheral tissue environment.

12. The method of claim 11, wherein the immune cells are blood dendritic cells and blood monocytes.

13. The method of claim 12, wherein the plurality of biological processes are combined so that the peripheral tissue environment is modeled with preferential recruitment of the blood dendritic cells over the blood monocytes.

14. The method of claim 10, wherein at least one biological process from the plurality of biological processes is associated with a biological variable that is a therapeutic agent.

15. The method of claim 10, further comprising:
producing a simulated biological attribute associated with the biological state of the adaptive immune response;
comparing the simulated biological attribute with a corresponding biological attribute in a reference pattern of the adaptive immune response; and
identifying the computer model as a valid computer model of the adaptive immune response if the simulated biological attribute is substantially consistent with the biological attribute associated with the reference pattern of the adaptive immune response.

16. The method of claim 10, wherein the combining the plurality of biological processes includes:
forming a first mathematical relation among biological variables associated with a first biological process from the plurality of biological processes; and
forming a second mathematical relation among biological variables associated with the first biological process and biological variables associated with a second biological process from the plurality of biological processes.

17. The method of claim 16, further comprising:
creating a set of parametric changes in the first mathematical relation and the second mathematical relation; and
producing a simulated biological attribute based on at least one parametric change from the set of parametric changes, the simulated biological attribute being substantially consistent with at least one biological attribute associated with a reference pattern of the adaptive immune response.

18. The method of claim 16, further comprising:
converting a first biological variable into a converted biological variable a value of which changes over time, the first biological variable being associated with at least one from the first mathematical relation and the second mathematical relation; and
producing a series of simulated biological attributes based on the converted biological variable, the series of simulated biological attributes being substantially consistent with a corresponding biological attribute associated with a reference pattern of the adaptive immune response, the series of simulated biological attributes representing the chronological progression of the corresponding biological attribute in the reference pattern of the adaptive immune response.

19. The method of claim 16, further comprising:
converting a parameter into a new biological variable a value of which changes over time, the parameter being associated with at least one from the first mathematical relation and the second mathematical relation; and
producing a series of simulated biological attributes based on the new biological variable, the series of simulated biological attributes being substantially consistent with a biological attribute associated with a reference pattern of an adaptive immune response, the series of simulated biological attributes representing the chronological progression of corresponding biological attributes in the reference pattern of the adaptive immune response.

20. A computer model of an adaptive immune response, comprising:
code to define a set of biological processes related to a biological state of the adaptive immune response; and
code to define a set of mathematical relationships related to interactions among biological variables associated with the set of biological processes, at least two biological processes from the set of biological processes being associated with the set of mathematical relationships, a combination of the code to define the set of biological processes and the code to define the set of mathematical relationships defining a simulation of the biological state of the adaptive immune response in the context of a peripheral tissue environment and a lymphoid tissue environment.

21. The computer model of claim 20, wherein at least one biological process from the set of biological processes is associated with recruitment of immune cells into the peripheral tissue environment.

22. The computer model of claim 21, wherein the immune cells are blood dendritic cells and blood monocytes.

23. The computer model of claim 22, wherein the set of biological processes are combined so that the peripheral tissue environment is modeled with preferential recruitment of the blood dendritic cells over the blood monocytes.

24. The computer model of claim 20, wherein at least one biological process from the set of biological processes is associated with a biological variable that is a therapeutic agent.

25. The computer model of claim 20, wherein upon execution of the code, a simulated biological attribute for the adaptive immune response is produced, the simulated biological attribute being substantially consistent with at least one biological attribute associated with a reference pattern of the adaptive immune response.

26. The computer model of claim 20, further comprising:
code to define a first compartment, said first compartment including biological processes related to a peripheral tissue environment, and
code to define a second compartment, said second compartment including biological processes related to a lymphoid tissue environment.

27. The computer model of claim 26, further comprising:
a code to define a set of biological processes related to trafficking of immune cells between said first and second compartments.

28. A computer executable software code, comprising:
code to define a plurality of biological processes related to a biological state of an adaptive immune response including:
code to define a set of mathematical relations associated with a first biological process from the plurality of biological processes and associated with interactions among biological variables associated with the first biological process, and
code to define a set of mathematical relations associated with a second biological process from the plurality of biological processes and associated with interactions among biological variables associated with the second biological process, the plurality of biological processes being associated with the adaptive immune response in the context of a peripheral tissue environment and a lymphoid tissue environment.

29. The computer executable software code of claim 28, wherein at least one biological process from the plurality of biological processes is associated with recruitment of immune cells into the peripheral tissue environment.

30. The computer executable software code of claim 29, wherein the immune cells are blood dendritic cells and blood monocytes.

31. The computer executable software code of claim 30, wherein the plurality of biological processes are combined so that the peripheral tissue environment is modeled with preferential recruitment of the blood dendritic cells over the blood monocytes.

32. The computer executable software code of claim 28, wherein at least one biological process from the plurality of biological processes is associated with a biological variable that is a therapeutic agent.

33. The computer executable software code of claim 28, further comprising:
code to receive a user selection of a link representation from a set of predefined link representations, each predefined link representation in the set of predefined link representations being uniquely associated with a mathematical relationship from the set of mathematical relationships, the user-selected link representation being associated with the interrelationship between a first biological variable and a second biological variable,
a first link representation from the set of predefined link representations being a representation of the first biological variable having an effect on the second biological variable,
a second link representation from the set of predefined link representations being a representation of instances of the first biological variable being converted to instances of the second biological variable.

34. The computer executable software code of claim 28, further comprising:
code to define a first compartment, said first compartment including biological processes related to a peripheral tissue environment, and
code to define a second compartment, said second compartment including biological processes related to a lymphoid tissue environment.

35. The computer executable software code of claim 34, further comprising:
a code to define a set of biological processes related to trafficking of immune cells between said first and second compartments.

36. A method for developing a computer model of an adaptive immune response, comprising:
receiving a plurality of user-selected indications to define a plurality of biological processes, each biological process from the plurality of biological processes being based on data that relates changes in a biological state of the adaptive immune response to biological attributes of a reference pattern of the adaptive immune response;
producing a simulated biological attribute associated with at least one biological attribute of the reference pattern of the adaptive immune response based on the combined plurality of biology processes; and
assessing validity of the computer model based on a comparison between the simulated biological attribute and a corresponding biological attribute associated with the reference pattern of the adaptive immune response.

37. A computer system of an adaptive immune response, comprising:
a computer-readable memory storing:
code to define a set of biological processes related to a biological state of the adaptive immune response; and
code to define a set of mathematical relationships related to interactions among biological variables associated with the biological processes, at least two biological processes from the set of biological processes being associated with the set of mathematical relationships, a combination of the code to define the set of biological processes and the code to define the set of mathematical relationships defining a simulation of the adaptive immune response in the context of a peripheral tissue environment and a lymphoid tissue environment; and
a processor coupled to the computer-readable memory, the processor being configured to execute the codes.

38. The computer system of claim 37, wherein at least one biological process from the set of biological processes is associated with a biological variable that is a therapeutic agent.

39. The computer system of claim 37, wherein upon execution of the code, a simulated biological attribute for the adaptive immune response is produced, the simulated biological attribute being substantially consistent with at least one biological attribute associated with a reference pattern of the adaptive immune response.

40. The computer system of claim 37, further comprising:
code to define a first compartment, said first compartment includes biological processes related to a peripheral tissue environment; and
code to define a second compartment, said second compartment includes biological processes related to a lymphoid tissue environment.

41. The computer system of claim 38, further comprising:
a code to define a set of biological processes related to trafficking of immune cells between said first and second compartments.

42. A method for developing an analytical model of an adaptive immune response, comprising:
identifying data relating to a biological state of the adaptive immune response;
identifying a plurality of biological processes related to the data, the plurality of biological processes defining at least one portion of the biological state of the adaptive immune response; and
combining the plurality of biological processes to form an analytical model of the adaptive immune response in the context of a peripheral tissue environment and a lymphoid tissue environment.

43. The method of claim 42, wherein at least one biological process from the plurality of biological processes is associated with recruitment of immune cells into the peripheral tissue environment.

44. The method of claim 43, wherein the immune cells are blood dendritic cells and blood monocytes.

45. The method of claim 44, wherein the plurality of biological processes are combined so that the peripheral tissue environment is modeled with preferential recruitment of the blood dendritic cells over the blood monocytes.

46. The method of claim 42, wherein the biological state of the adaptive immune response is a biological state of an acute response.

47. The method of claim 42, wherein the biological state of the adaptive immune response is a biological state of a chronic response.

48. The method of claim 42, further comprising:
producing an analytical representation of a biological attribute associated with the adaptive immune response;
comparing the analytical representation of the biological attribute with a corresponding biological attribute in a reference pattern of the adaptive immune response; and
identifying the analytical model as a valid model of the adaptive immune response if the analytical representation of the biological attribute is substantially consistent with the biological attribute associated with the reference pattern of the adaptive immune response.

49. The method of claim 42, wherein the combining the plurality of biological processes includes:
forming a first mathematical relation among biological variables associated with a first biological process from the plurality of biological processes; and
forming a second mathematical relation among biological variables associated with the first biological process and biological variables associated with a second biological process from the plurality of biological processes.

50. The method of claim 42, wherein at least one biological process from the plurality of biological processes is associated with a biological variable that is a therapeutic agent.

51. A method for developing a computer model of the biological state of an antigen-presenting cell, comprising:
identifying data relating to a plurality of physiological regulatory mechanisms of the antigen-presenting cell, the data being associated with at least two from the group of antigen processing, migration, maturation, and mediator production of the antigen-presenting cell;
identifying a plurality of biological processes related to the data, the plurality of biological processes defining at least one portion of the role of the antigen-presenting cell in an adaptive immune response; and
combining the plurality of biological processes to form a simulation of the functioning of the antigen-presenting cell in context of the adaptive immune response.

52. The method of claim 51, wherein at least one biological process from the plurality of biological processes is associated with a biological variable that is a therapeutic agent.

53. The method of claim 51, wherein the antigen-presenting cell is a dendritic cell.

54. The method of claim 53, wherein the dendritic cell is a myeloid dendritic cell.

55. The method of claim 51, wherein at least one biological process from the plurality of biological processes is associated with a differential response of lymphocytes to antigen based on the maturational state of the antigen-presenting cell.
